(19) United States
(12) Patent Application Publication
(10) Pub. No.: US 2010/0308305 A1
(43) Pub. Date: Dec. 9, 2010

(54) ORGANIC LUMINESCENT MATERIAL AND ORGANIC LIGHT EMITTING DEVICE USING THE SAME

(75) Inventors: Hyo Nim Shin, Seoul (KR); Chi Sik Kim, Seoul (KR); Young Jun Cho, Seoul (KR); Hyuck Joo Kwon, Seoul (KR); Bong Ok Kim, Seoul (KR); Sung Min Kim, Seoul (KR); Seung Soo Yoon, Seoul (KR)

Correspondence Address: ROHM AND HAAS ELECTRONIC MATERIALS LLC, 455 FOREST STREET, MARLBOROUGH, MA, 01752, US

(73) Assignee: Gracel Display Inc., Seoul (KR)

(21) Appl. No.: 12/452,660

(22) PCT Filed: Jul. 3, 2008
(86) PCT No.: PCT/KR2008/003917

§ 371 Date: Aug. 19, 2010

(30) Foreign Application Priority Data
Jul. 13, 2007 (KR) 1020070070614

(51) Int. Cl. H01L 51/54; C07C 13/48; C07C 13/465; C07C 25/22; C07F 7/08

(52) U.S. Cl. 257/40; 585/26; 585/27; 570/129; 556/489; 257/E51.041

(57) ABSTRACT

The present invention relates to novel organic electroluminescent materials and organic light emitting devices comprising the same. Since the organic electroluminescent materials according to the invention have good luminous efficiency and life property as an electroluminescent material, OLED's having very good operation lifetime can be produced.

### TECHNICAL FIELD

The present invention relates to organic electroluminescent materials having the structure represented by Chemical Formula (1) and organic light emitting devices comprising the same.

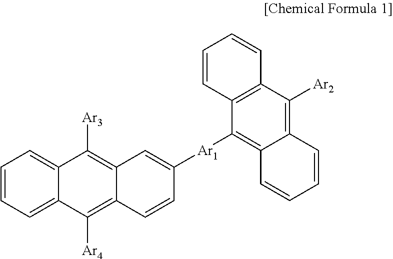

wherein, Ar₁ is a (C₅-C₂₀) aromatic ring or a fused polycyclic aromatic ring with two or more aromatic rings having been fused, provided that Ar₁ is not anthracenyl;

Ar₂ through Ar₄ independently represent a (C₅-C₂₀) aromatic ring or a fused polycyclic aromatic ring with two or more aromatic rings having been fused; and

the aromatic ring or the fused polycyclic aromatic ring with two or more aromatic rings having been fused of Ar₁ through Ar₄ may be further substituted by one or more substituent(s) selected from (C₁-C₂₀)alkyl, (C₁-C₂₀)alkoxy, halogen, tri(C₁-C₂₀)alkylsilyl, tri(C₆-C₂₀)arylsilyl, a (C₅-C₂₀) aromatic ring and a fused polycyclic aromatic ring with two or more aromatic rings having been fused.

### BACKGROUND ART

The most important factor to develop an organic electroluminescent (EL) device having high efficiency and long lifetime is development of an electroluminescent material having high performances.

In case of blue light, it becomes advantageous from the aspect of the luminous efficiency, if the light emitting wavelength is shifted a little toward longer wavelength. However, it is not easy to apply the material to a display of high quality because of unsatisfactory pure blue color. In addition, there are problems of color purity, efficiency and thermal stability.

For blue materials, a number of materials have been developed and commercialized since the development of DPVBi (Chemical Formula a) was disclosed in European Patent Laid-Open Publication No. 1063869 by Idemitsu-Kosan Company Limited. The distyryl compound system by Idemitsu-Kosan, which has been known to have the highest efficiency up to now, has 6 lm/W of power efficiency and beneficial device lifetime of more than 30,000 hr. However, when it is applied to a full-colored display, owing to the reduction of color purity over operation time, the lifetime is merely several thousand hours.

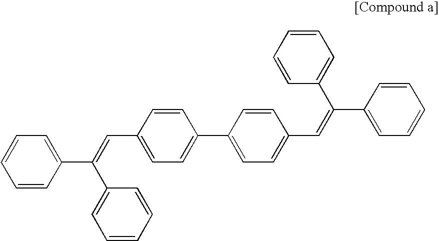

In the meanwhile, the dinaphthylanthracene compound (Compound b) disclosed in U.S. Pat. No. 6,465,115 by Kodak is claimed as HTL material, which has been also utilized as a blue electroluminescent compound. However, the compound still has problems to be solved in view of luminous efficiency and color purity.

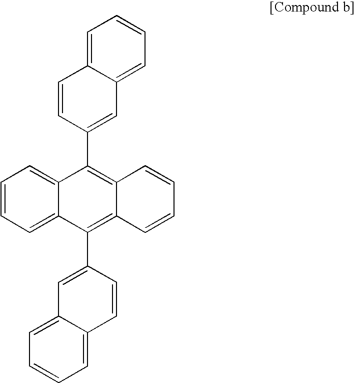

Recently, the electroluminescent derivatives (Compound c) within the similar scope of Compound (b) have been disclosed in WO2006/25700 by LG Chem. However, those Compounds (c) also have limitations in luminous efficiency and color purity.

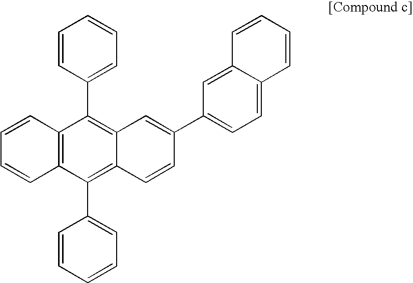

In the meanwhile, as a green fluorescent material, a system wherein a coumarine derivative (Compound d, C545T), a quinacridone derivative (Compound e), DPT (Compound f) or the like is doped as a dopant to Alq (a host), in a concentration from several % to not more than 20% has been developed and widely used. However, the conventional electroluminescent materials suffer from significant problem in view of lifetime with noticeable reduction of initial efficiency, though they show good performance in view of initial luminous efficiency at the level of practical use. Thus, the materials have limitations to be employed for a high performance panel of larger screen.

It has been reported that this is resulted from short life of cationic species of Alq which was used as a host. In order to overcome the problem, development of a host with amphoteric property, which simultaneously has stability to cationic species and anionic species, is very urgent.

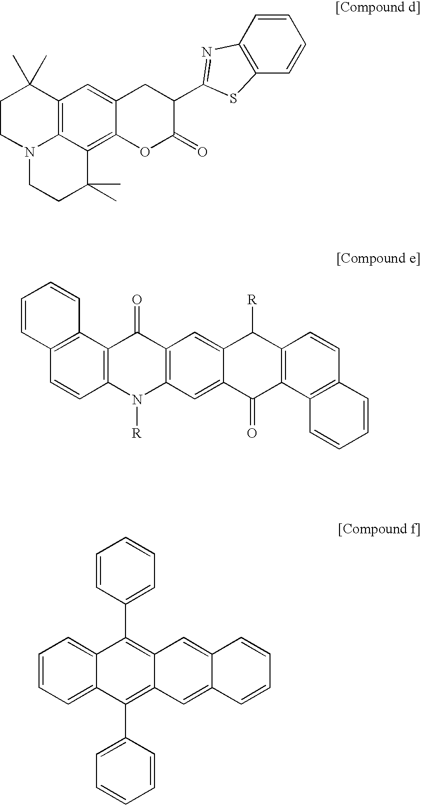

### DISCLOSURE

### Technical Problem

The object of the invention is to overcome the problems as described above, and to provide electroluminescent compounds having noticeably improved properties of the host which serves as a solvent or an energy carrier in the electroluminescent material, as compared to those of the conventional materials. In addition, the object of the invention is to provide a blue or green electroluminescent material with improved luminous efficiency and lifetime of the device, and an organic light emitting device comprising the same.

### Technical Solution

The present invention relates to organic electroluminescent compounds represented by Chemical Formula 1:

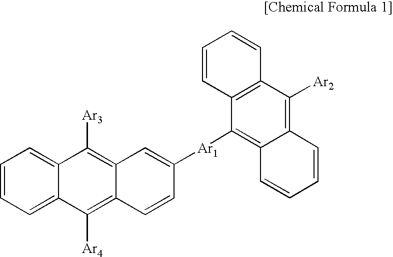

wherein, Ar₁ is a (C₅-C₂₀) aromatic ring or a fused polycyclic aromatic ring with two or more aromatic rings having been fused, provided that Ar₁ is not anthracenyl;

Ar₂ through Ar₄ independently represent a (C₅-C₂₀) aromatic ring or a fused polycyclic aromatic ring with two or more aromatic rings having been fused; and

the aromatic ring or the fused polycyclic aromatic ring with two or more aromatic rings having been fused of Ar₁ through Ar₄ may be further substituted by one or more substituent (s) selected from (C₁-C₂₀)alkyl, (C₁-C₂₀)alkoxy, halogen, tri(C₁-C₂₀)alkylsilyl, tri(C₆-C₂₀)arylsilyl, a (C₅-C₂₀) aromatic ring and a fused polycyclic aromatic ring with two or more aromatic rings having been fused.

Since the organic electrolumescent material according to the invention has good luminous efficiency and life property as an electroluminescent material, OLED's having very good operation lifetime can be produced.

The electroluminescent materials mentioned in the present specification include, in a broad sense, any material employed as the organic substance in an organic light emitting device comprised of a first electrode, a second electrode and an organic substance interposed between the first and the second electrode; while they imply, in a narrow sense, what is applied to an electroluminescent host which serves as an electroluminescent medium in an electroluminescent layer.

In the compound represented by Chemical Formula (1) according to the present invention, Ar₁ represents phenylene, biphenylene, naphthylene, fluorenylene, spirobifluorenylene, phenanthrylene, triphenylenylene, pyrenylene, chrysenylene or naphthacenylene, and Ar₁ may be further substituted by (C₁-C₂₀)alkyl or phenyl; Ar₂ through Ar₄ independently represent phenyl, naphthyl, anthryl, biphenyl, fluorenyl, phenanthryl, triphenylenyl, pyrenyl, chrysenyl or naphthacenyl, and Ar₂ through Ar₄ may be further substituted by one or more substitutent (s) selected from (C₁-C₂₀)alkyl, (C₁-C₂₀)alkoxy, halogen, tri (C₁-C₂₀)alkylsilyl, tri(C₆-C₂₀)arylsilyl, phenyl, naphthyl, anthryl, fluorenyl, 9,9-dimethyl-fluoren-2-yl and 9,9-diphenyl-fluoren-2-yl.

The organic electroluminescent material represented by Chemical Formula (1) according to the present invention may be specifically exemplified by the following compounds, without being restricted thereto.

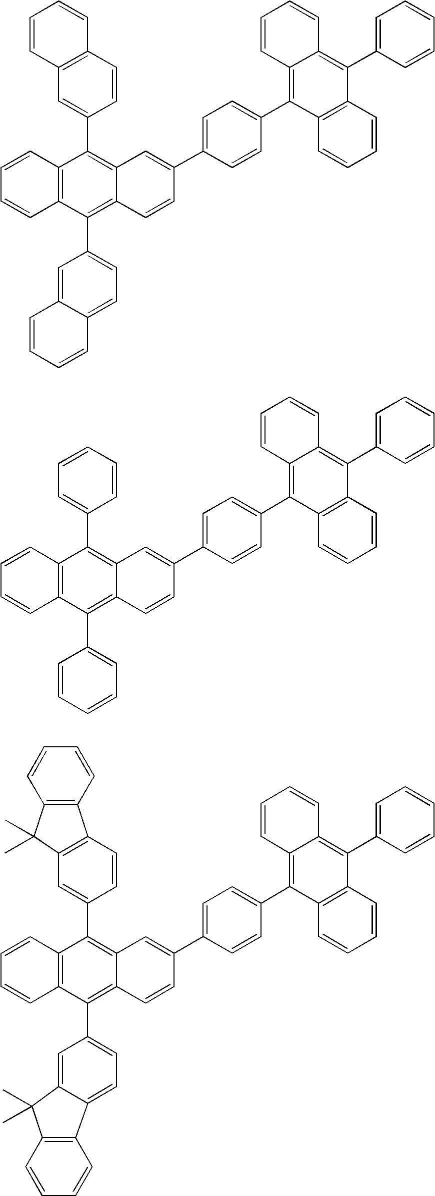

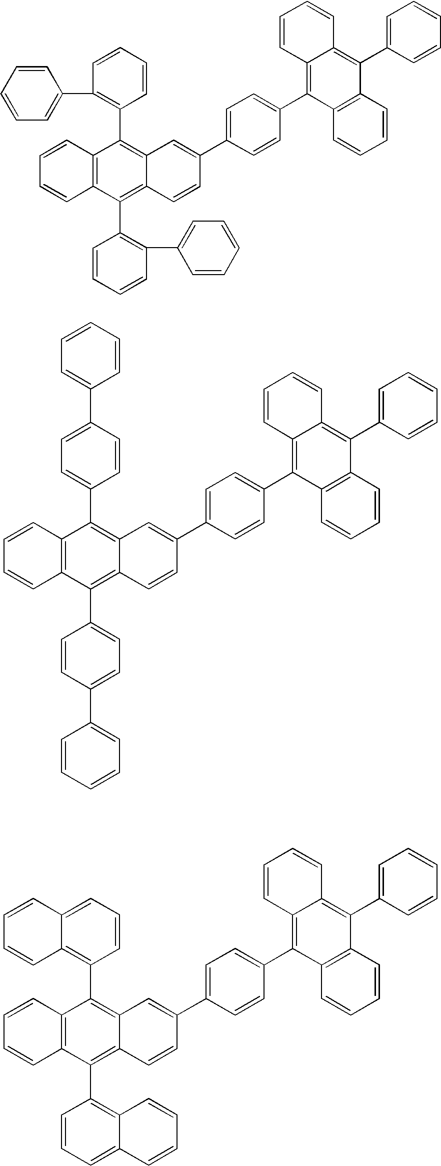

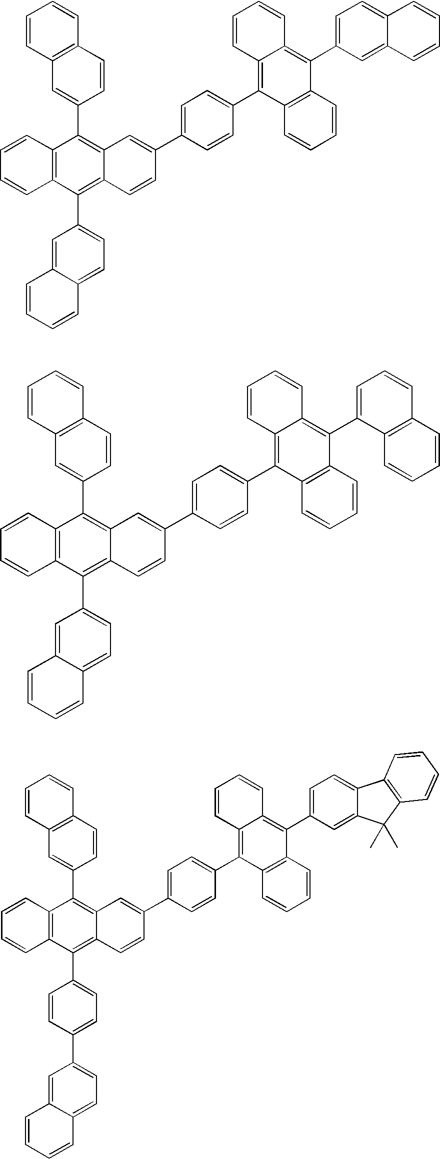

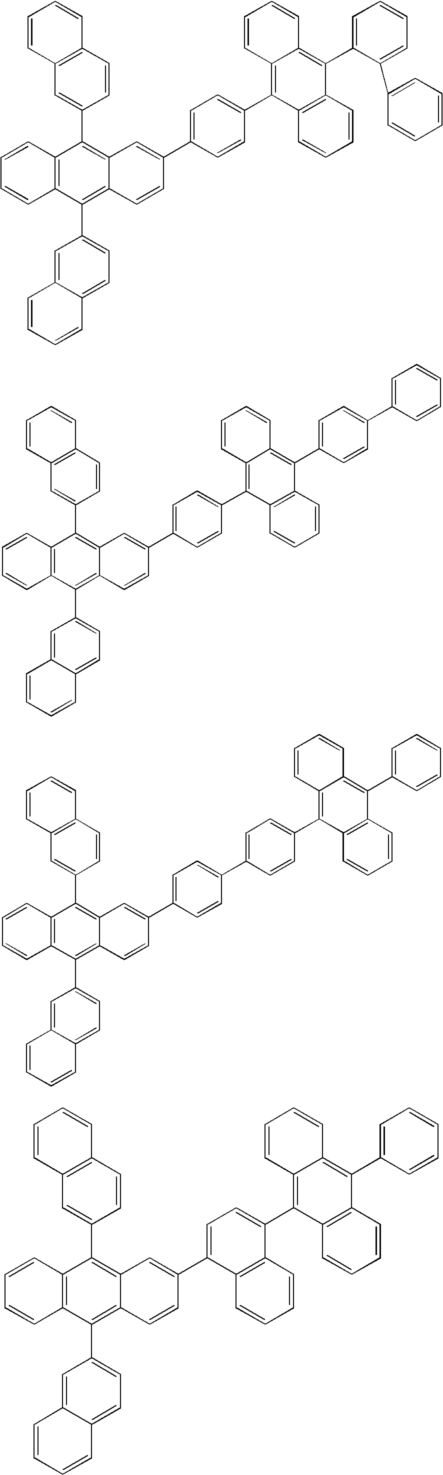

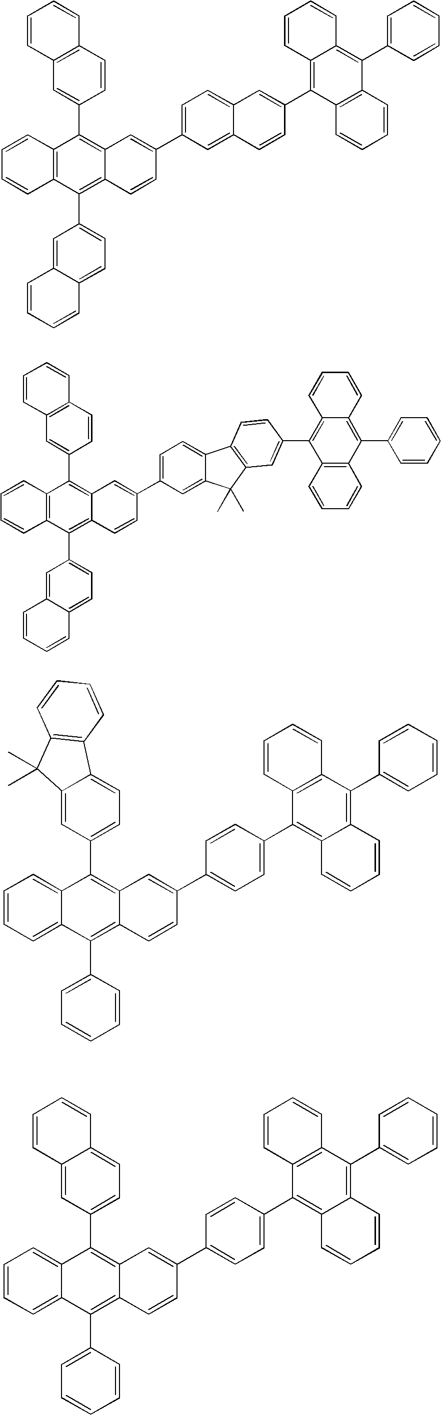

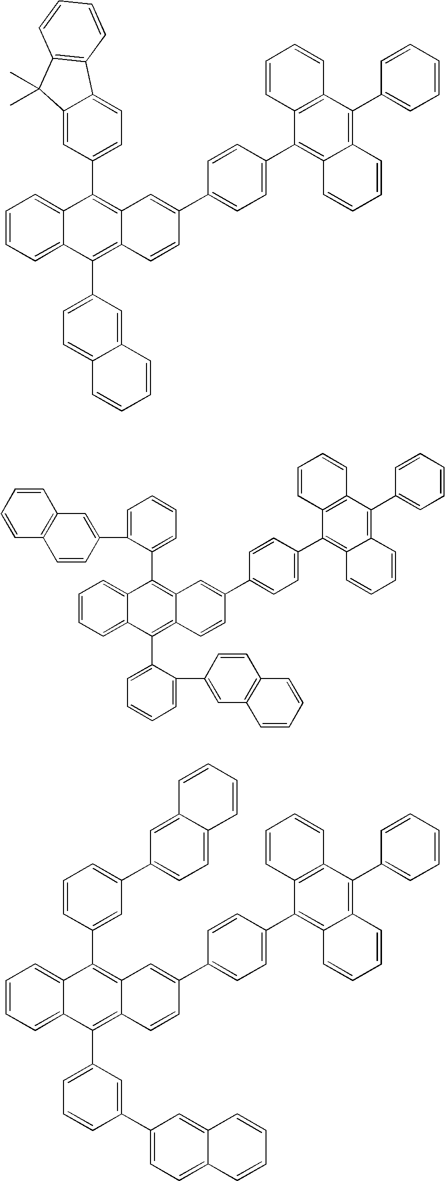

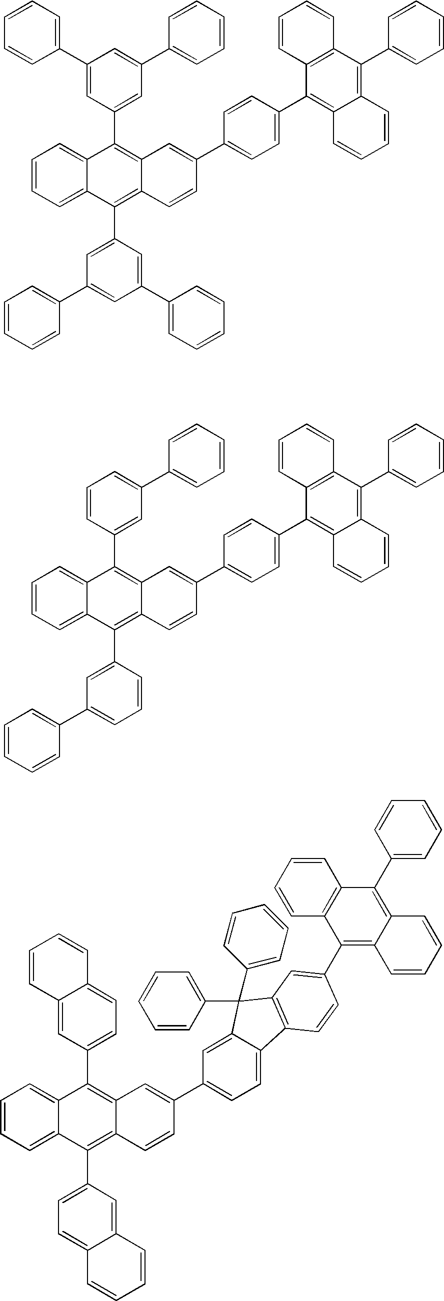

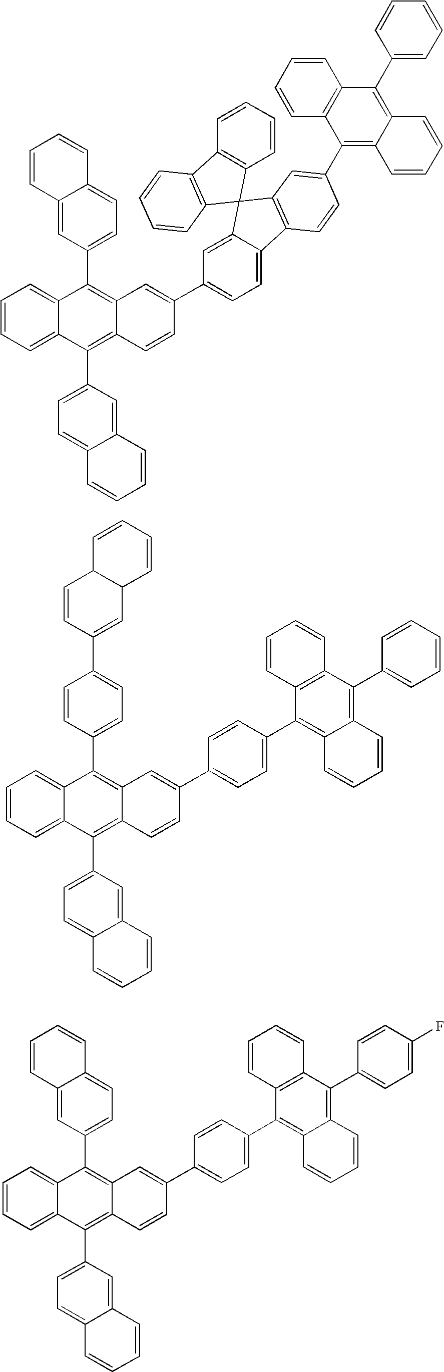

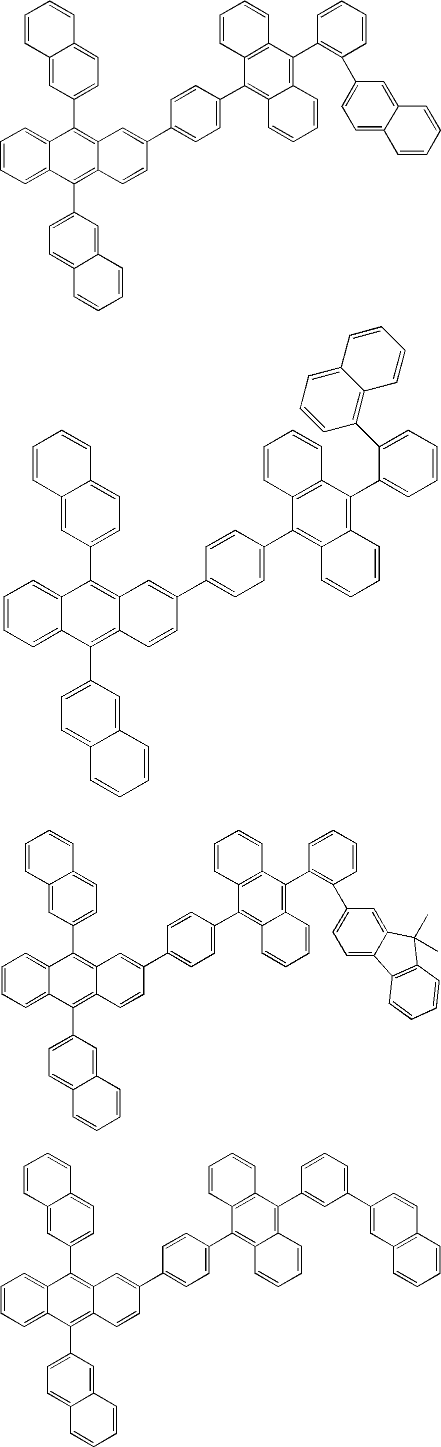

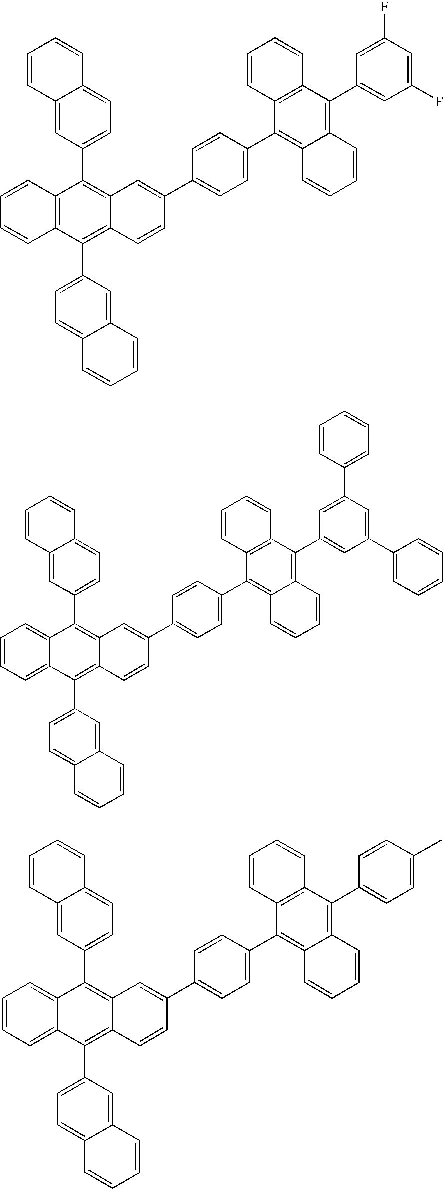

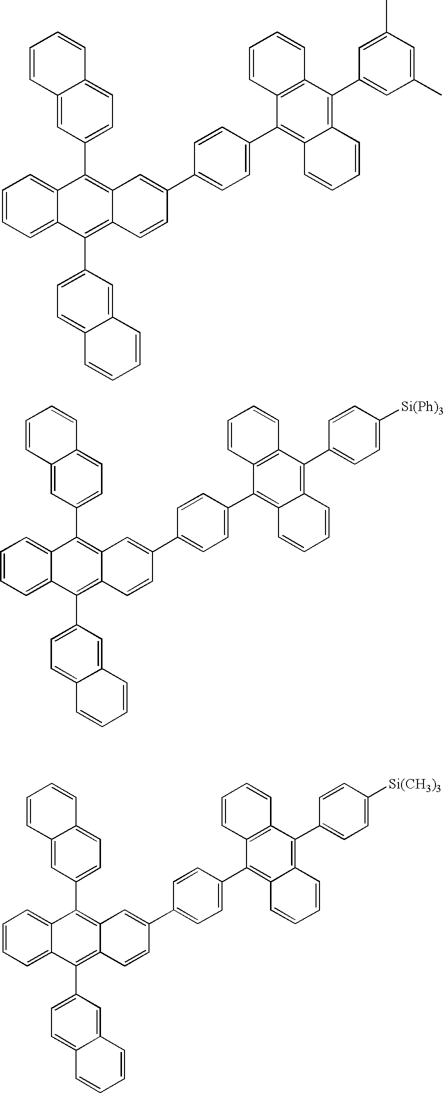

The present invention also provides an organic light emitting device comprised of a first electrode; a second electrode; and one or more organic layer(s) interposed between the first electrode and the second electrode, wherein the organic layer comprises one or more compound(s) represented by Chemical Formula (1):

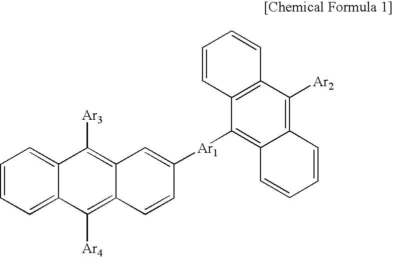

wherein, Ar₁ is a (C₅-C₂₀) aromatic ring or a fused polycyclic aromatic ring with two or more aromatic rings having been fused, provided that Ar₁ is not anthracenyl;

Ar₂ through Ar₄ independently represent a (C₅-C₂₀) aromatic ring or a fused polycyclic aromatic ring with two or more aromatic rings having been fused; and

the aromatic ring or the fused polycyclic aromatic ring with two or more aromatic rings having been fused of Ar₁ through Ar₄ may be further substituted by one or more substituent (s) selected from (C₁-C₂₀)alkyl, (C₁-C₂₀)alkoxy, halogen, tri (C₁-C₂₀)alkylsilyl, tri(C₆-C₂₀)arylsilyl, a (C₅-C₂₀) aromatic ring and a fused polycyclic aromatic ring with two or more aromatic rings having been fused.

The organic light emitting device according to the present invention is characterized in that the organic layer comprises EL region which comprises one or more EL dopant with one or more compound(s) represented by Chemical Formula (1) as an EL host. The EL dopants applied to the organic light emitting device of the invention are not particularly restricted, but exemplified, in case of blue color, by the compounds represented by one of Chemical Formulas (2) to (4):

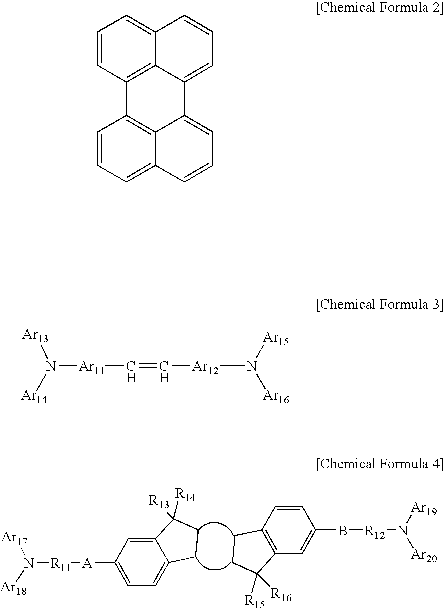

In the Chemical Formula (3) or (4), Ar₁₁ and Ar₁₂ are independently selected from indenofluorenylene, fluorenylene and spiro-fluorenylene, represented by following chemical formulas:

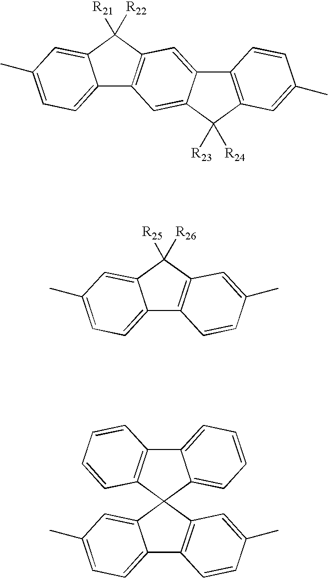

wherein Ar₁₃ through Ar₁₆ are independently selected from (C₅-C₂₀) aromatic or polycyclic aromatic rings; provided that Ar₁₁ and Ar₁₂ are identical, Ar₁₃ and A₁₅ are identical, and Ar₁₄ and Ar₁₆ are identical.

Ar₁₇ through Ar₂₀ independently represent a (C₅-C₂₀) aromatic ring or a fused polycyclic aromatic ring with two or more aromatic rings having been fused;

group

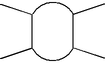

represents

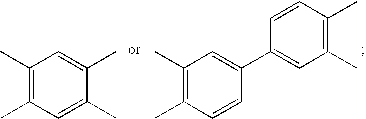

A and B independently represent a chemical bond, or group

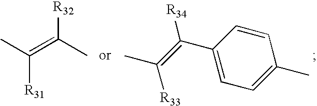

R₁₁ and R₁₂ independently represent a (C₅-C₂₀) aromatic ring or a polycyclic aromatic ring with two or more aromatic rings having been fused;

R₁₃ through R₁₆ independently represent a linear or branched (C₁-C₂₀) alkyl group with or without halogen substituent(s);

R₂₁ through R₂₆ are independently selected from (C₁-C₂₀) alkyl, and phenyl or naphthyl with or without (C₁-C₅)alkyl substituent(s); and

R₃₁ through R₃₄ independently represent hydrogen or a (C₅-C₂₀) aromatic group.

The compounds of Chemical Formula (3) or (4) may be specifically exemplified by the compounds represented by one of the following formulas:

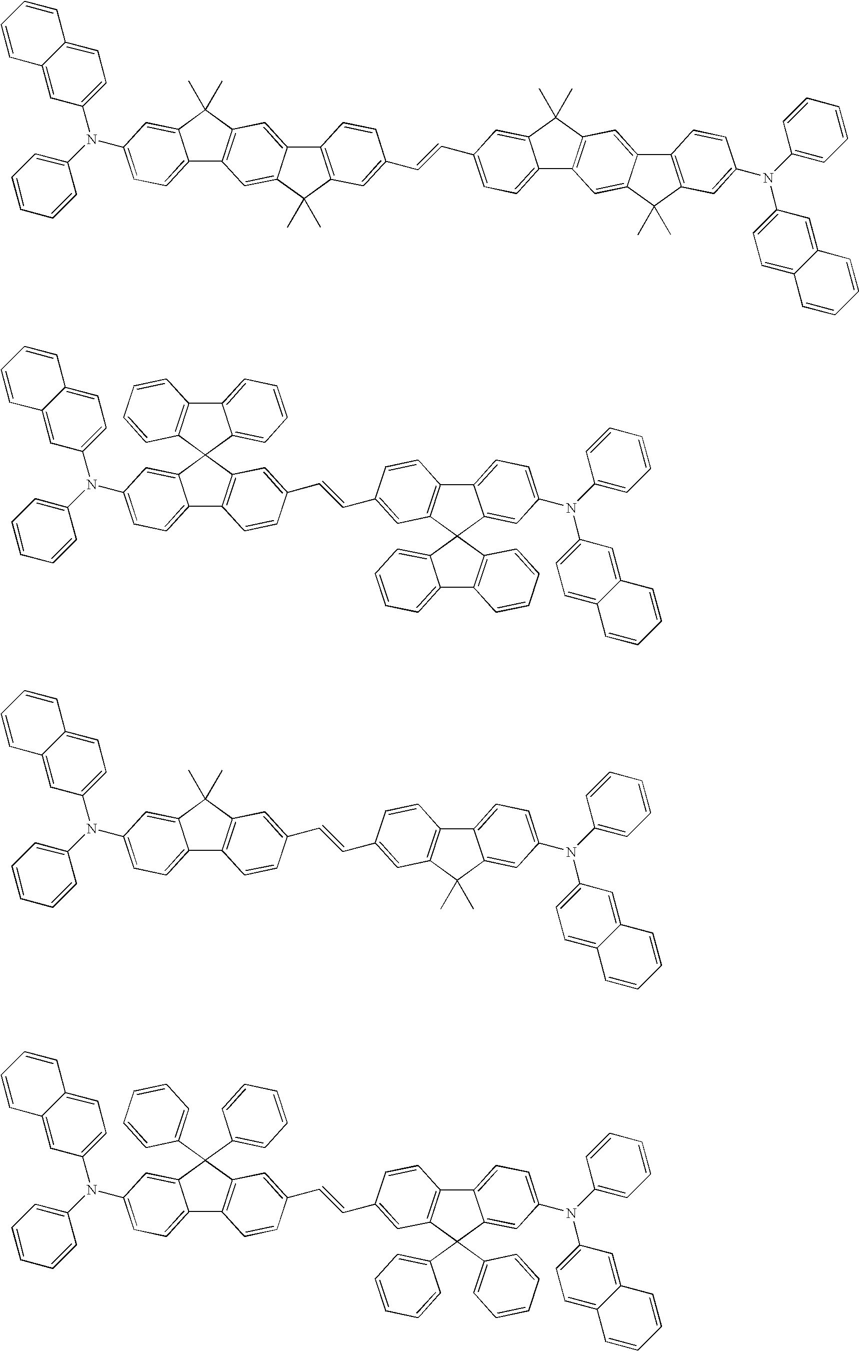

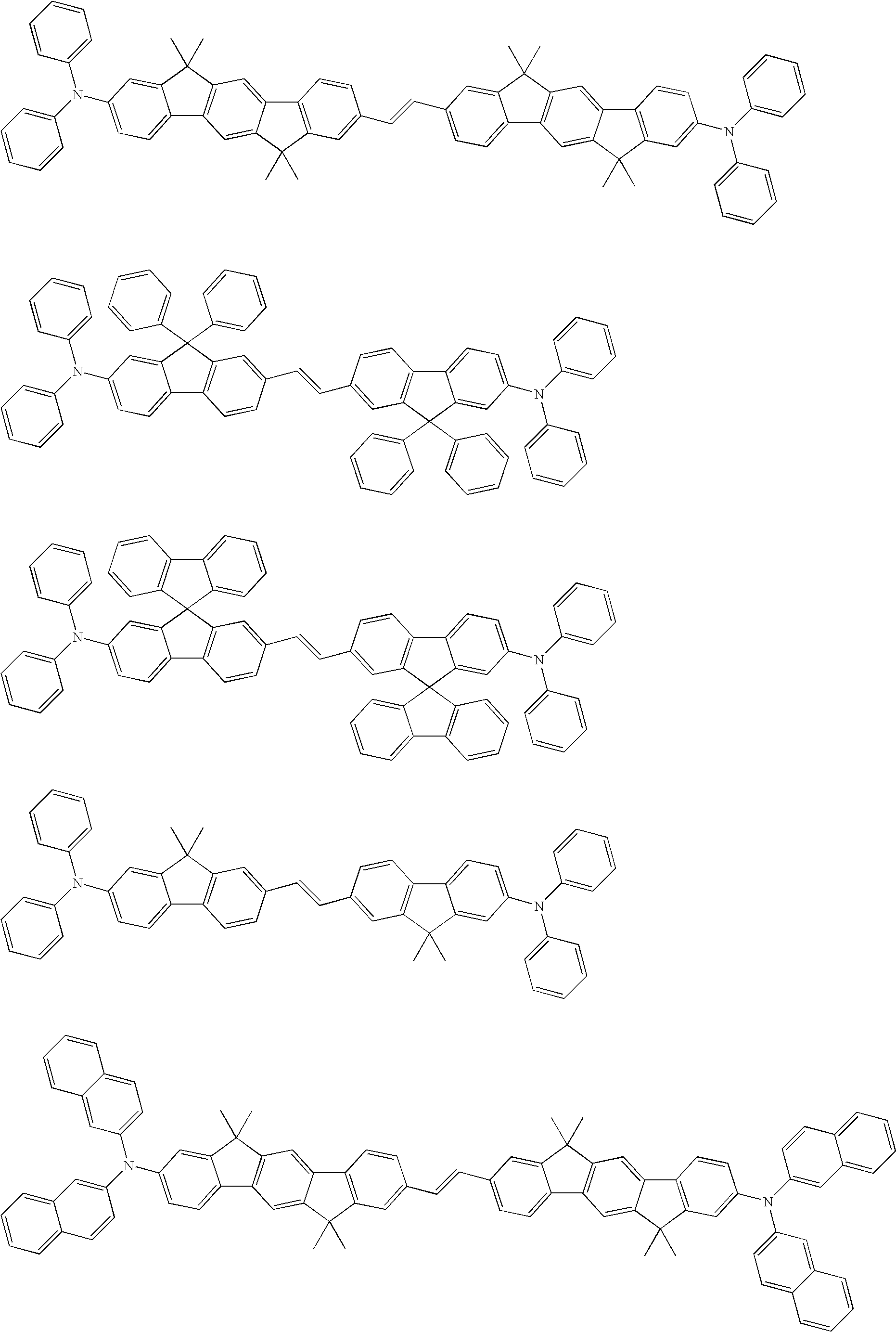

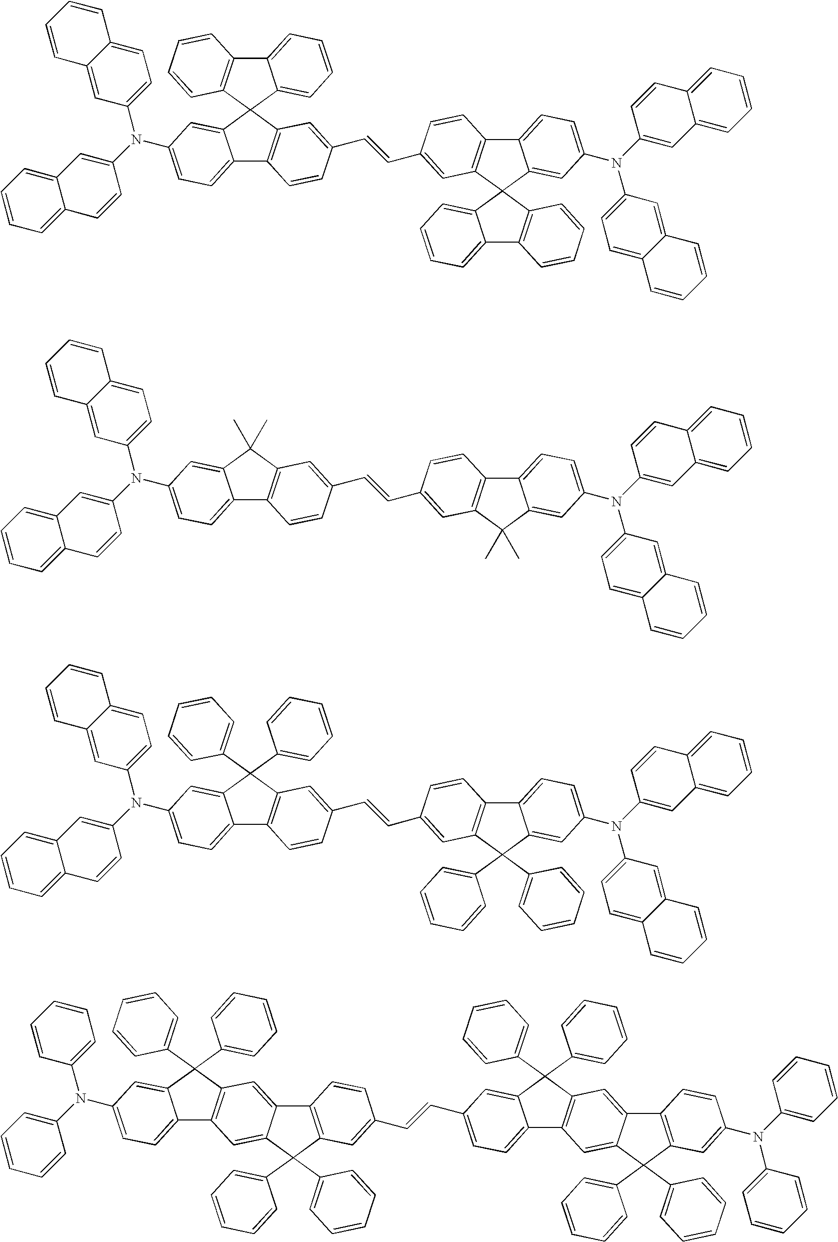

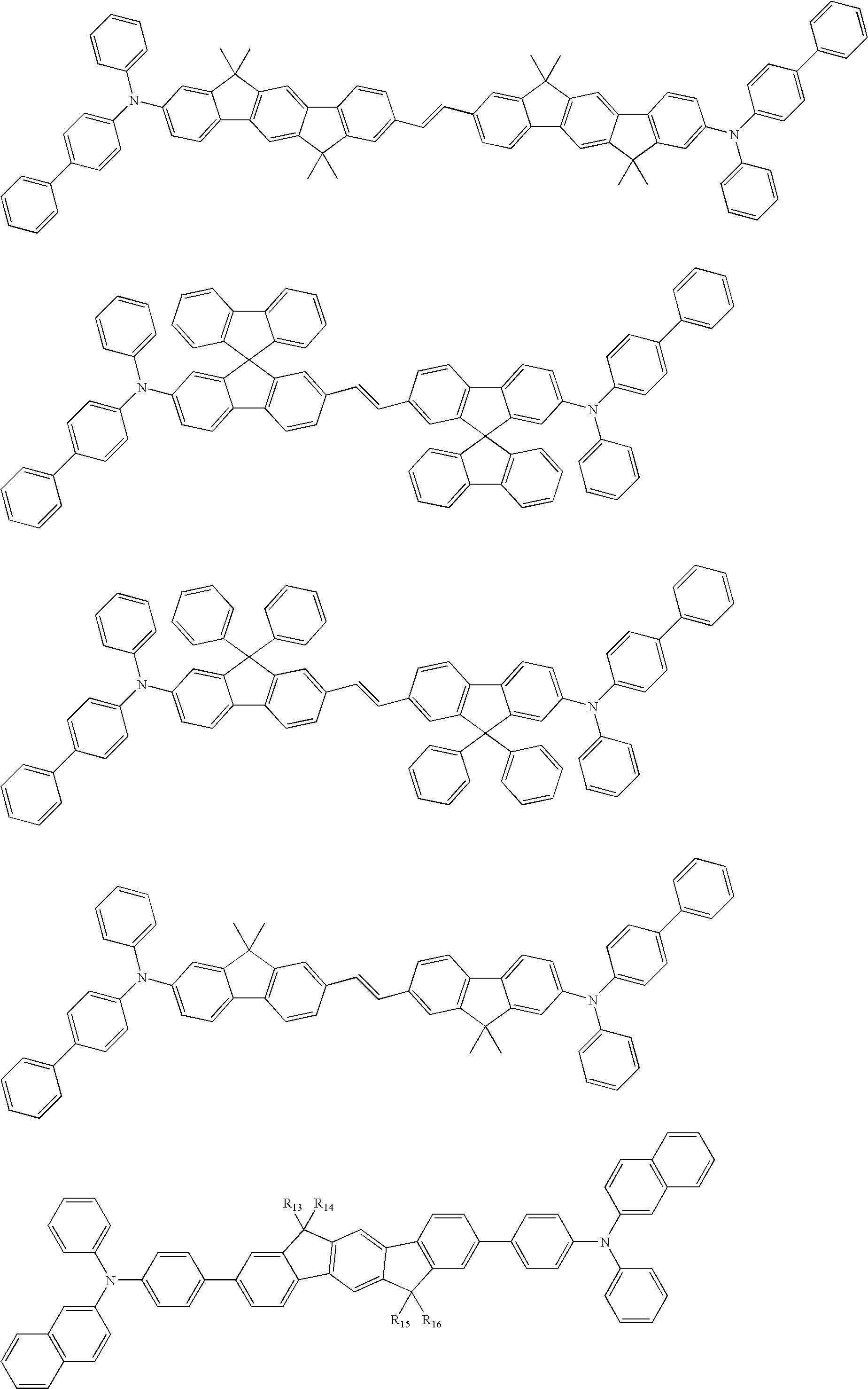

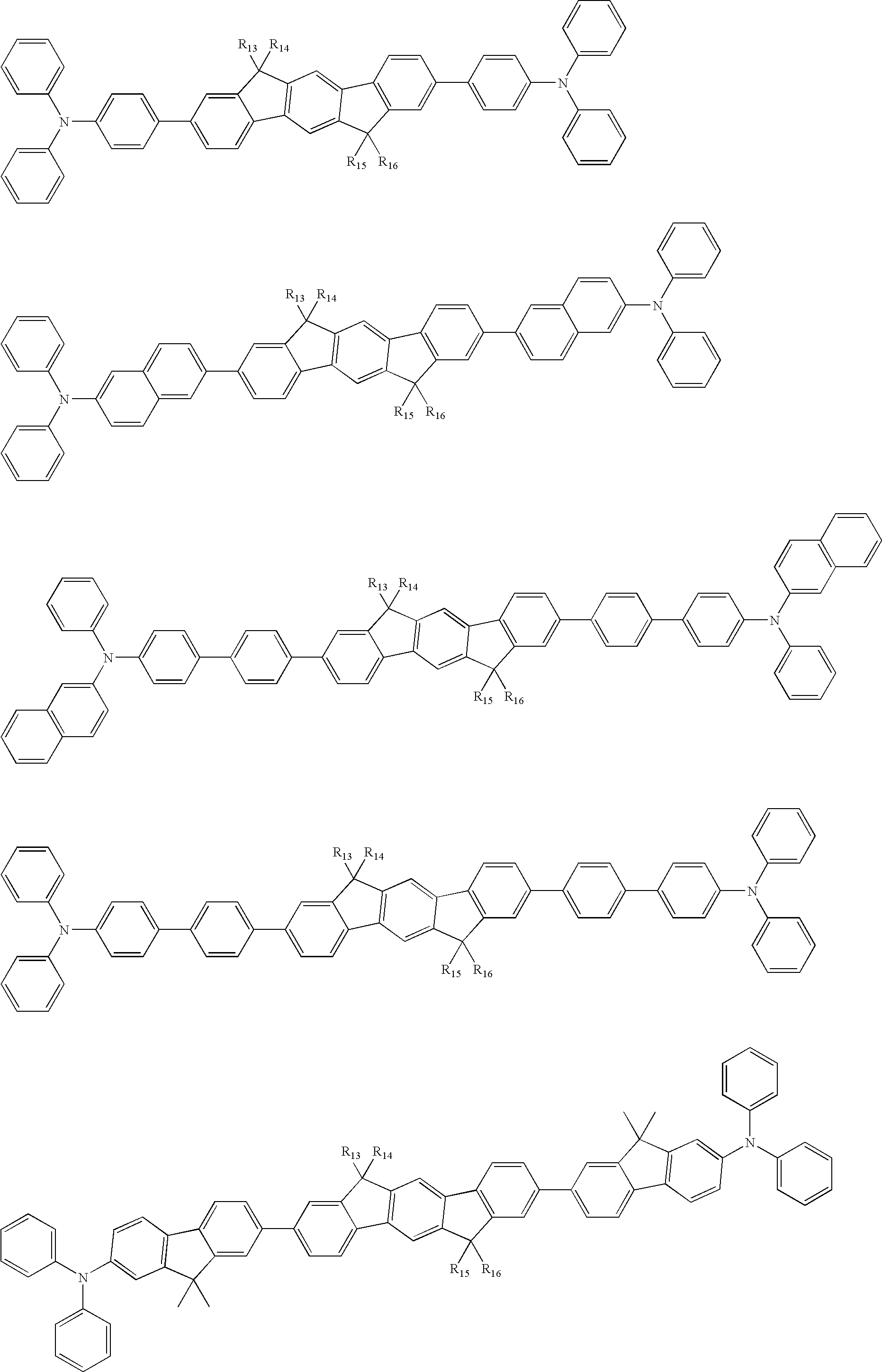

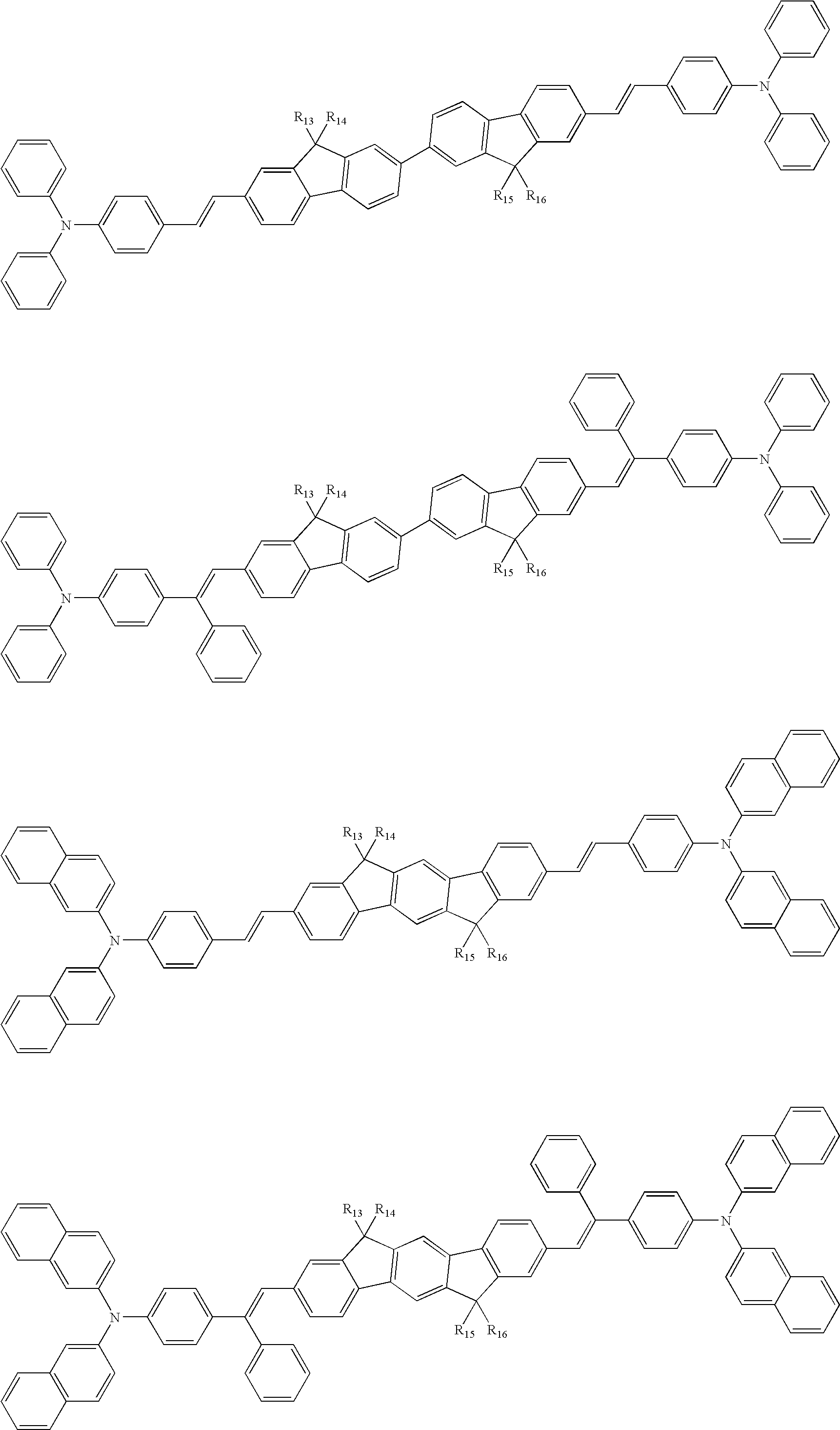

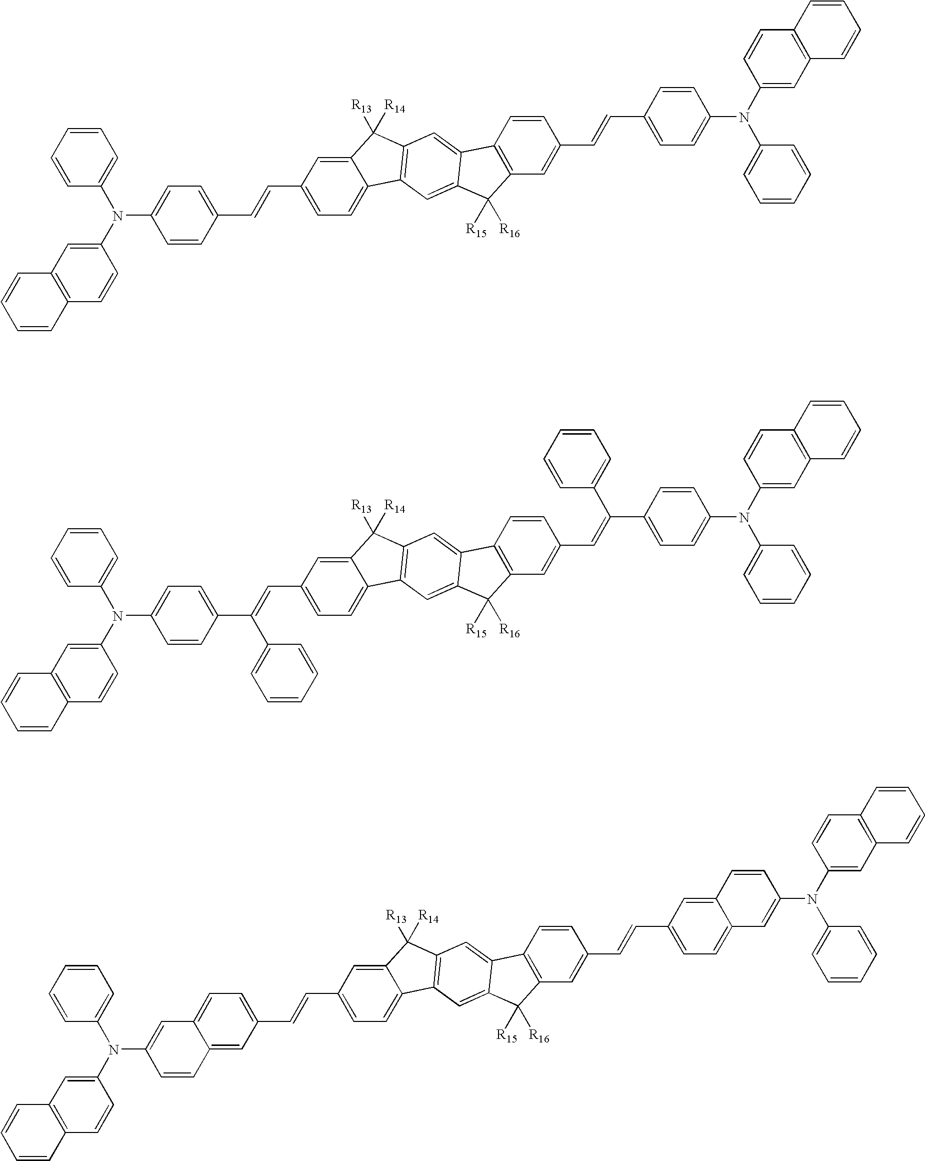

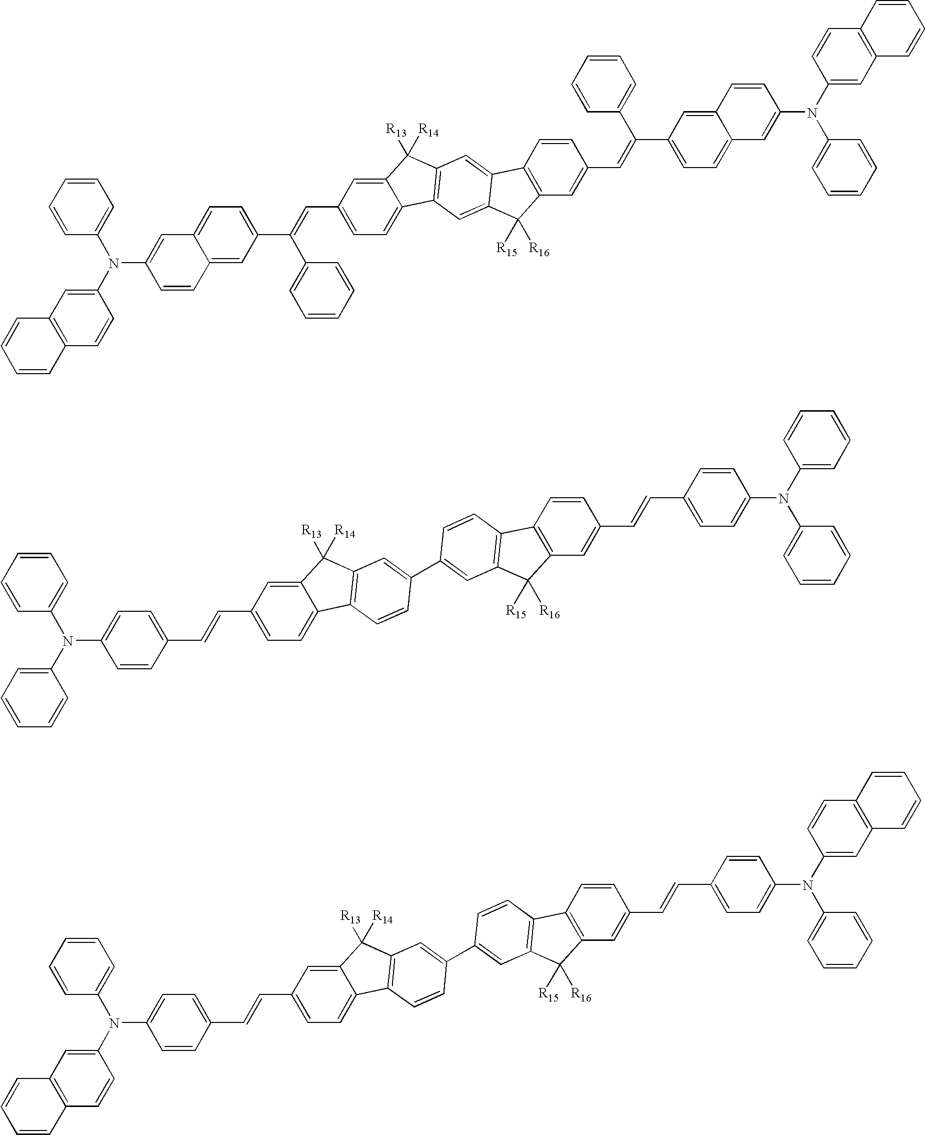

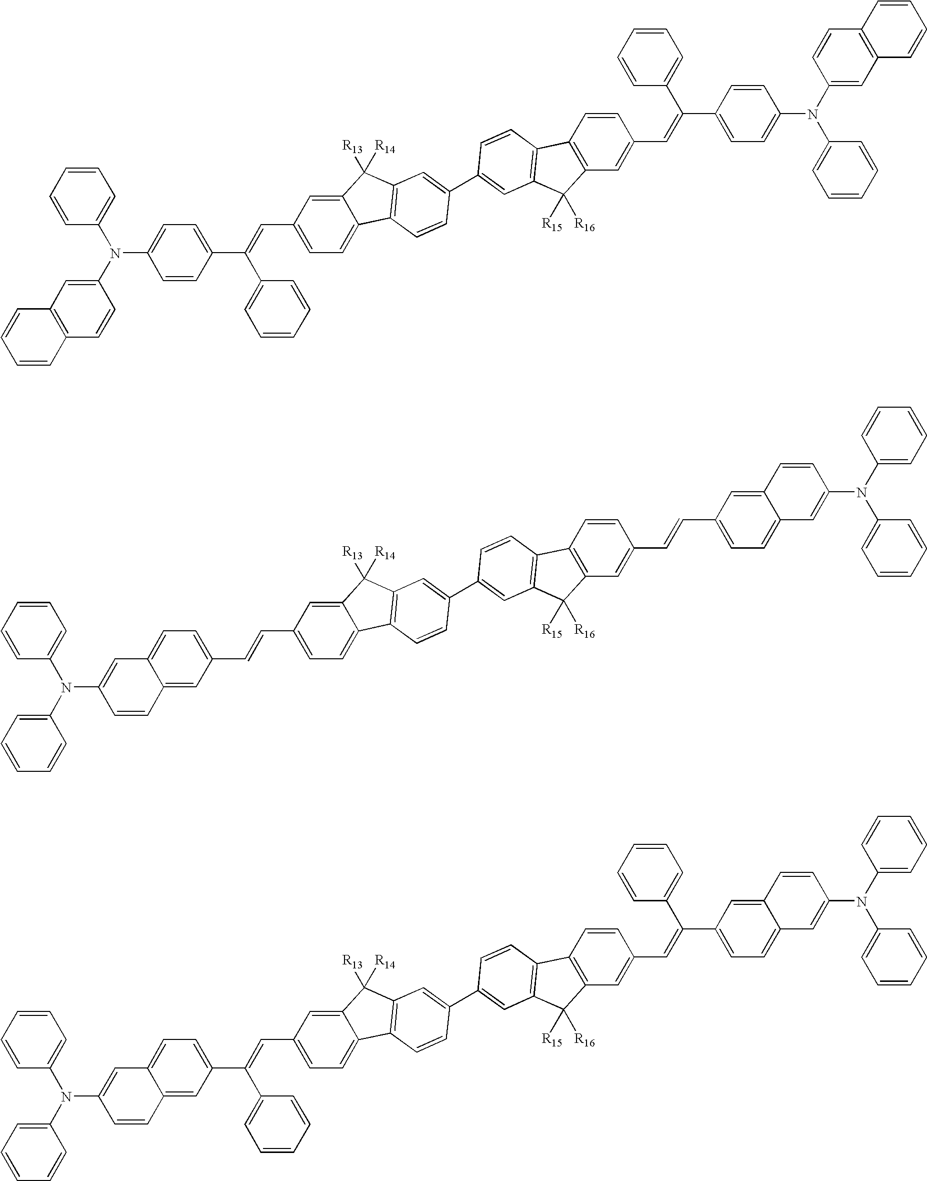

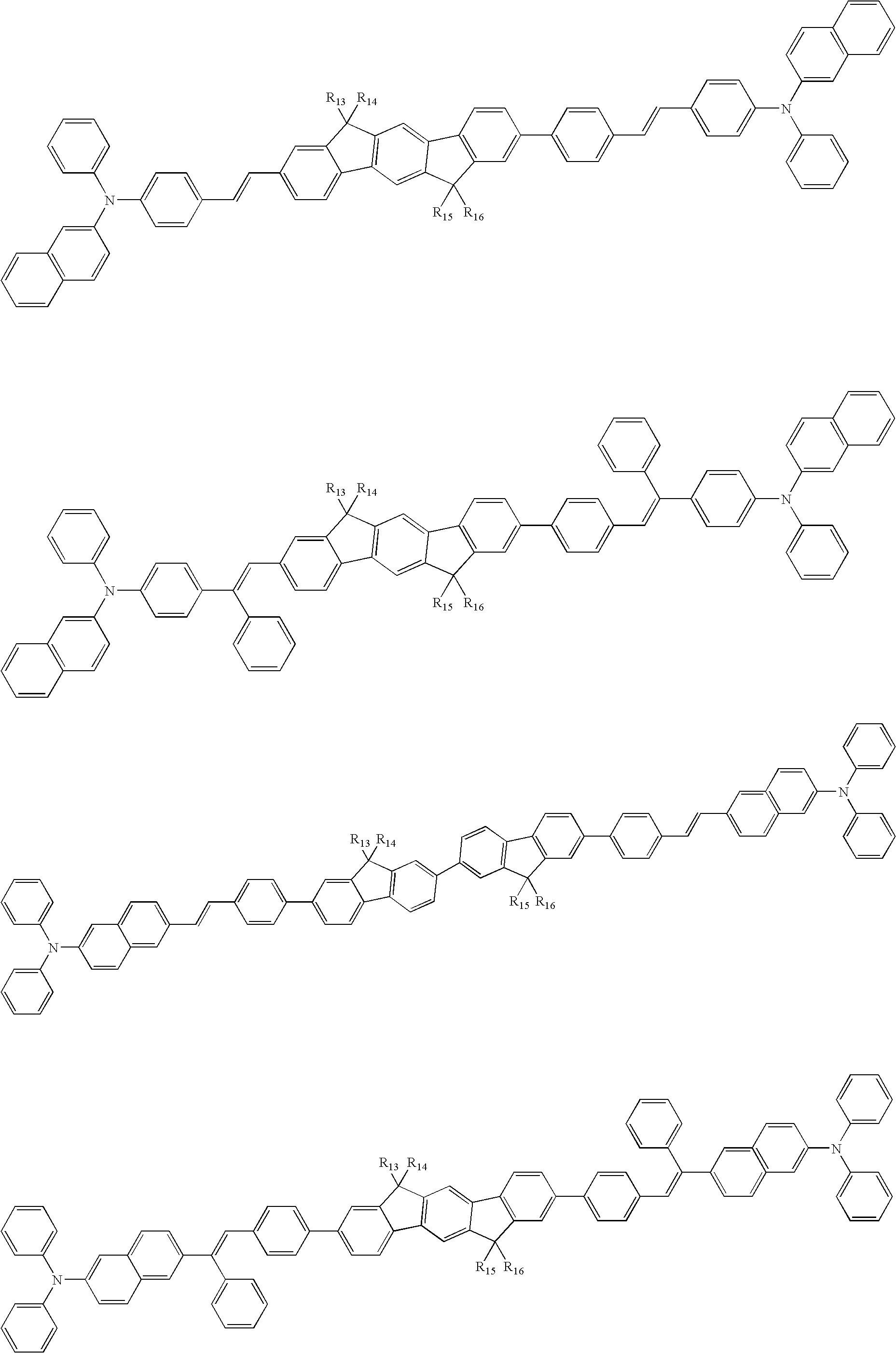

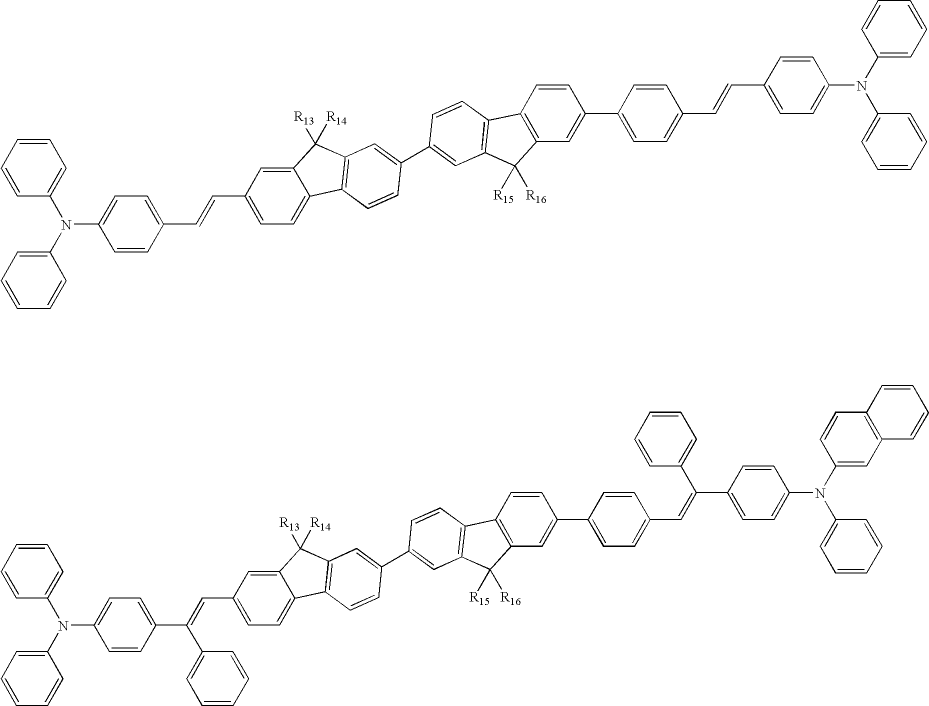

wherein, R₁₃ through R₁₆ independently represent methyl or ethyl group.

For green EL material, the electroluminescent dopant can be exemplified by the compounds selected from one of Chemical Formulas (5) to (7):

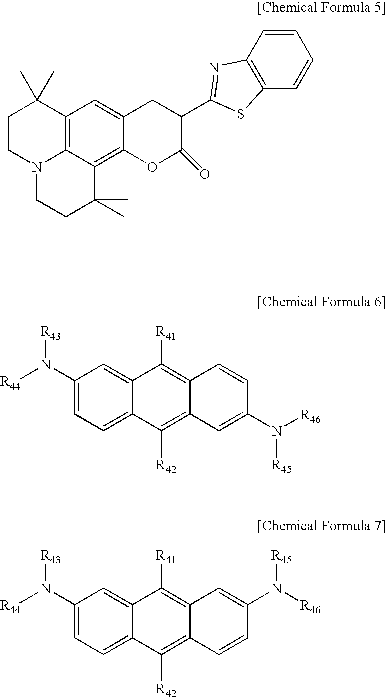

In Chemical Formulas (6) or (7), R₄₁ and R₄₂ independently represent a polycyclic aromatic ring with two more (C₅-C₂₀) aromatic rings having been fused; R₄₃ through R₄₆ independently represent a (C₅-C₂₀) aromatic ring; and each aromatic ring of R₄₁ through R₄₆ may be further substituted by (C₁-C₂₀) alkyl or (C₅-C₂₀) aryl group(s).

The compounds of Chemical Formulas (6) or (7) can be specifically exemplified by the compounds represented by one of the following structures:

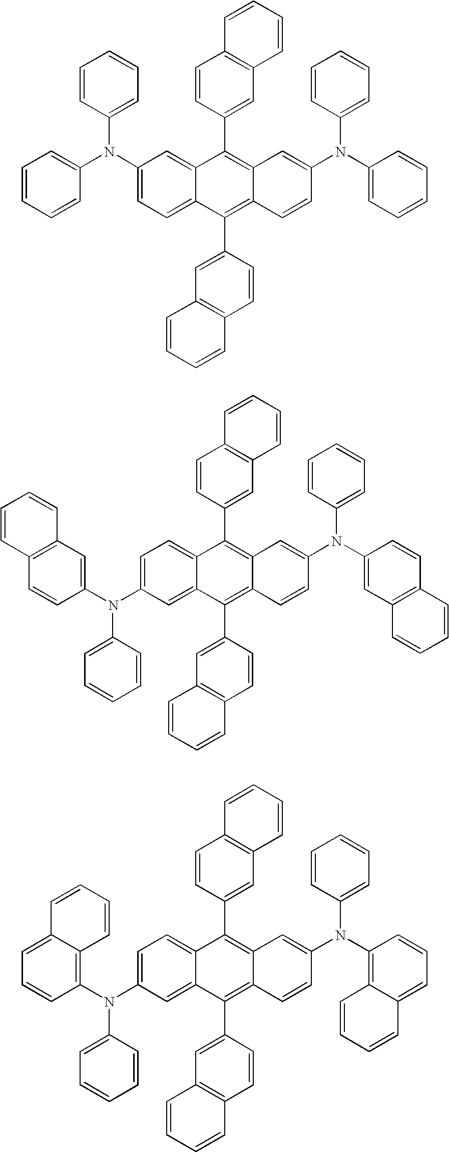

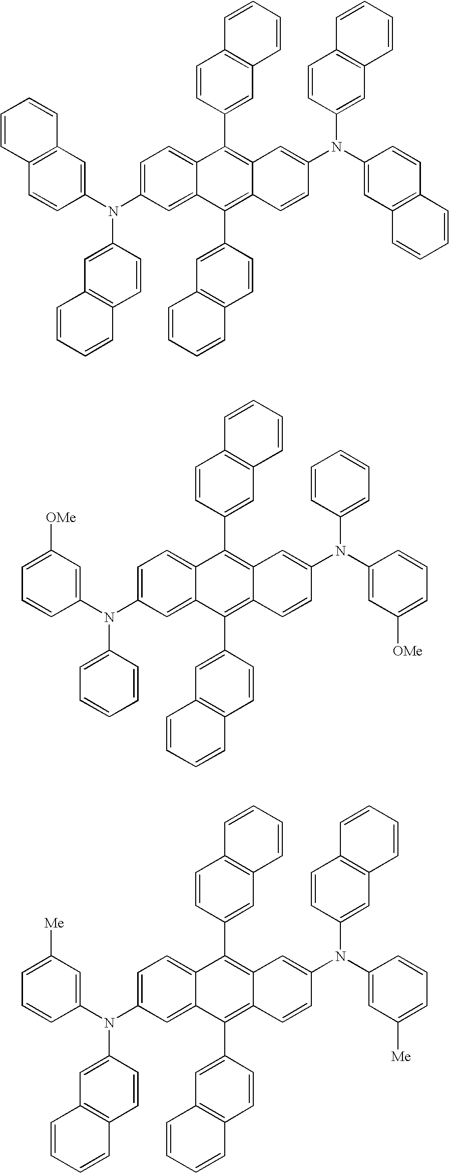

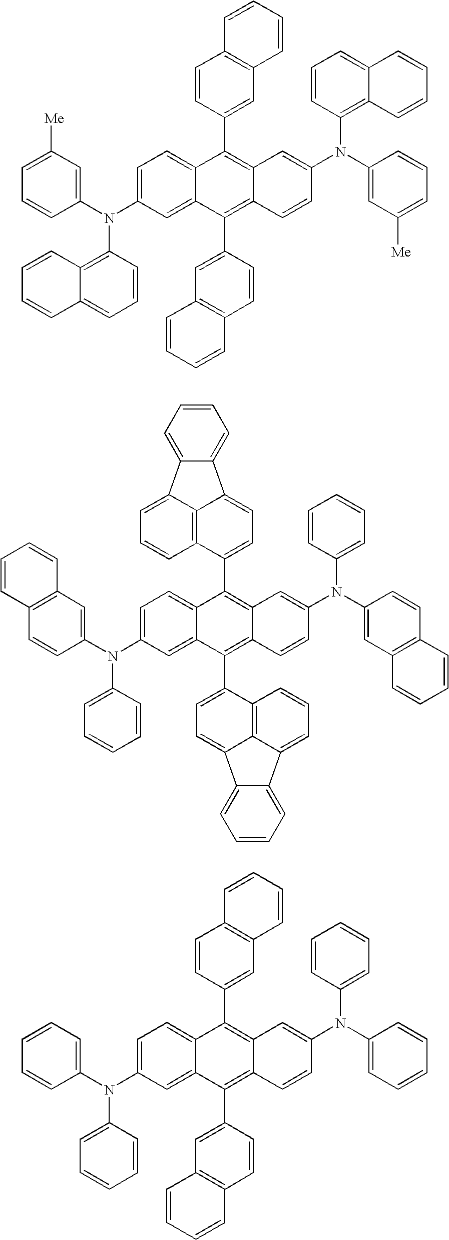

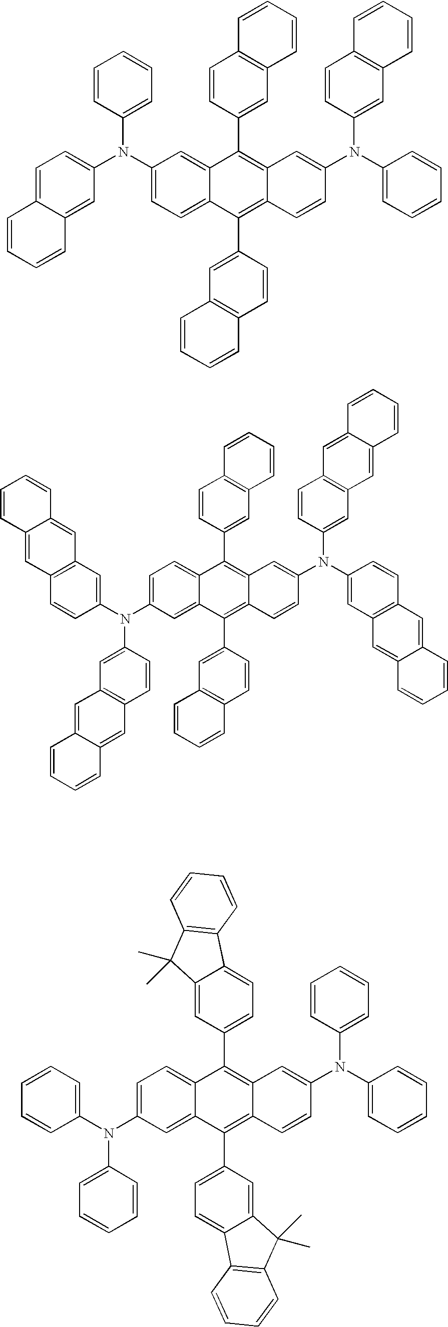

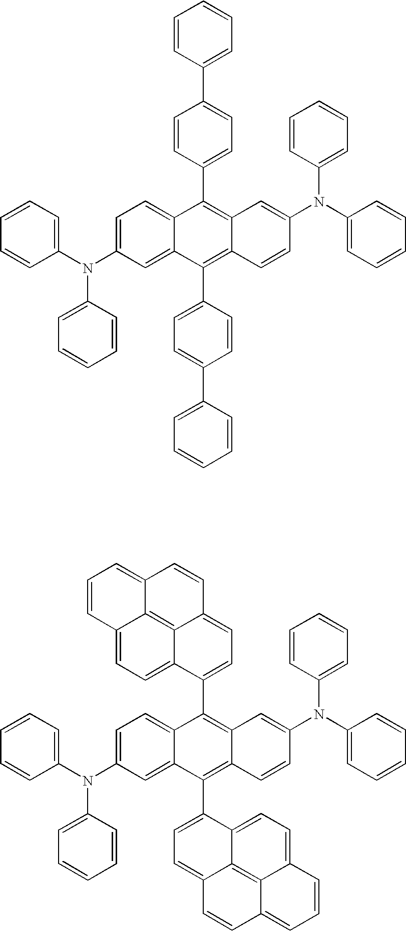

### BRIEF DESCRIPTION OF DRAWINGS

FIG. 1 is a cross-sectional view of an OLED;

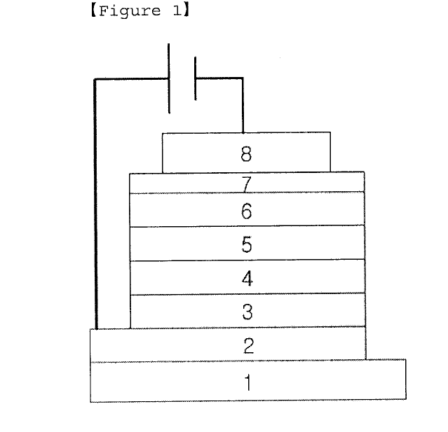

### DESCRIPTION OF SYMBOLS OF SIGNIFICANT PARTS OF THE DRAWINGS

1: Glass
2: Transparent electrode
3: Hole injection layer
4: Hole transportation layer
5: Electroluminescent layer
6: Electron transportation layer
7: Electron injection layer
8: Al cathode

### ADVANTAGEOUS EFFECTS

Since the organic electroluminescent compounds according to the invention have good luminous efficiency and life property as an electroluminescent material, OLED's having very good operation lifetime can be produced.

### BEST MODE

The present invention is further described with respect to the processes for preparing organic electroluminescent compounds according to the invention by referring to Examples, which are provided for illustration only but are not intended to limit the scope of the invention by any means.

### Preparation Examples

### Preparation of Compound of Chemical Formula (1)

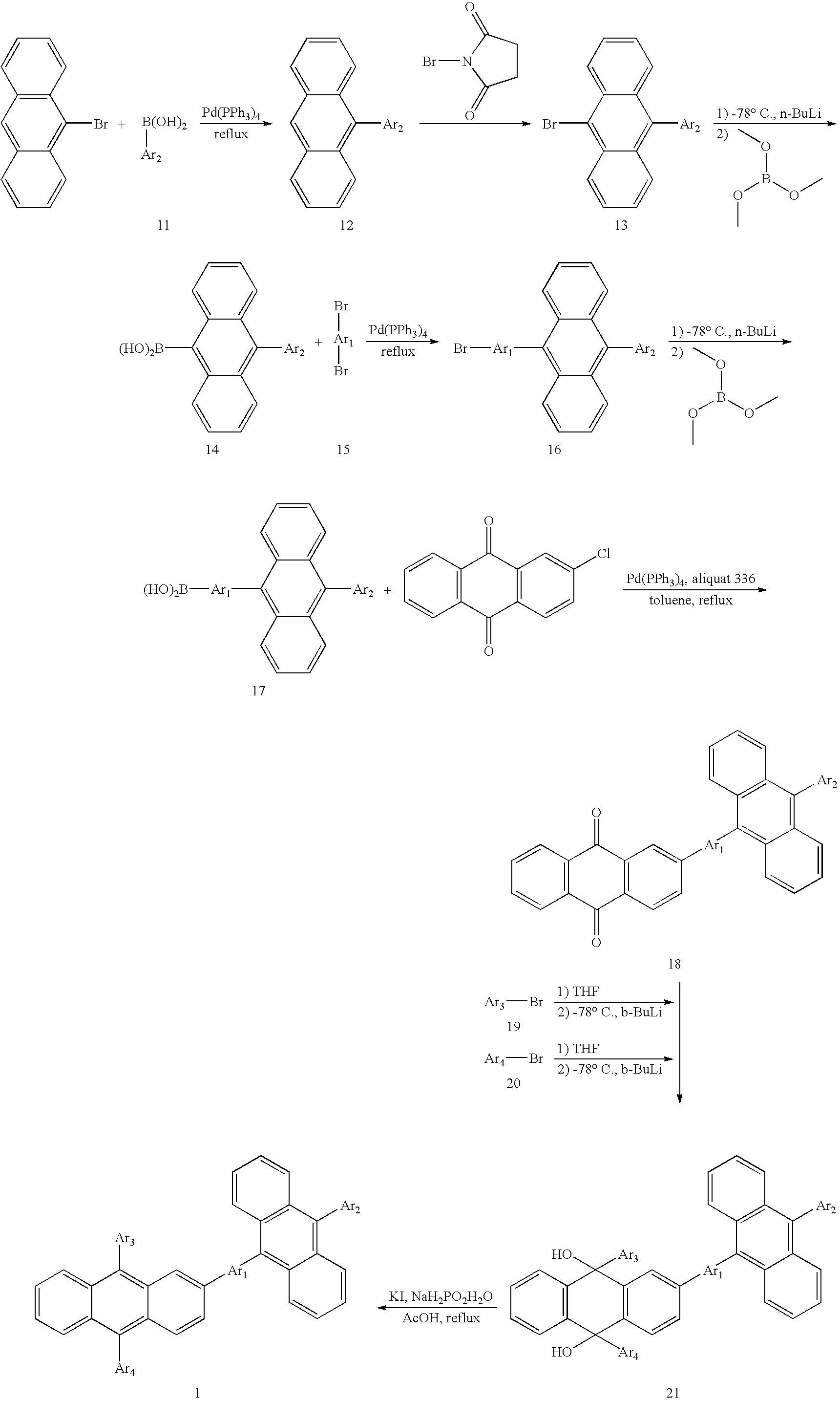

### Preparation of Compound (12)

In a mixed solution of toluene and ethanol (2:1 by volume), dissolved were 9-bromoanthracene (58.3 mmol), boronic derivative (Compound II) (70.0 mmol) and tetrakis palladium (0) triphenylphosphine (Pd(PPh₃)₄) (5.8 mmol). Aqueous 2M sodium carbonate solution was added to the solution, and the mixture was stirred at 120° C. under reflux for 5 hours. Then the temperature was lowered to 25° C., and distilled water was added thereto to quench the reaction. The reaction mixture was extracted with ethyl acetate, and the extract was dried under reduced pressure. Recrystallization from tetrahydrofuran and methanol gave Compound (12).

### Preparation of Compound (13)

Compound (12) (46.0 mmol) obtained as above and N-bromosuccinimide (50.6 mmol) were dissolved in dichloromethane under nitrogen atmosphere, and the solution was stirred at 25° C. for 5 hours. Then distilled water was added to quench the reaction. The reaction mixture was extracted with dichloromethane, and the extract was dried under reduced pressure. Recrystallization from tetrahydrofuran and methanol gave Compound (13).

### Preparation of Compound (14)

Compound (13) (39.0 mmol) obtained as above was dissolved atmosphere, and the solution was chilled to −78° C. To the solution, n-butyl lithium (1.6 M in hexane) (46.8 mmol) was slowly added dropwise, and the mixture was stirred for 1 hour. Then trimethylborate (78.0 mmol) was added thereto. The temperature was slowly raised, and the reaction mixture was stirred at 25° C. for one day. Aqueous 1M HCl solution was added thereto, and the resultant mixture was stirred at ambient temperature. After quenching the reaction, the mixture was extracted with ethyl acetate, and the extract was dried under reduced pressure. Recrystallization from methylene chloride and hexane gave Compound (14).

### Preparation of Compound (16)

In a mixed solution of toluene and ethanol (2:1 by volume), dissolved were Compound (14) (32.1 mmol) obtained as above, the dibromo derivative of Compound (15) (32.1 mmol) and tetrakis palladium (0) triphenylphosphine (Pd(PPh₃)₄) (3.2 mmol). Aqueous 2M sodium carbonate solution was added to the solution, and the mixture was stirred at 120° C. under reflux for hours. Then the temperature was lowered to 25° C., and distilled water was added thereto to quench the reaction. The reaction mixture was extracted with ethyl acetate, and the extract was dried under reduced pressure. Recrystallization from tetrahydrofuran and methanol gave Compound (16).

### Preparation of Compound (17)

Compound (16) (26.1 mmol) obtained as above was dissolved in thoroughly purified tetrahydrofuran under nitrogen atmosphere, and the solution was chilled to −78° C. To the solution, n-butyl lithium (1.6 M in hexane) (31.3 mmol) was slowly added dropwise, and the mixture was stirred for 1 hour. Then trimethylborate (31.3 mmol) was added thereto. The temperature was slowly raised, and the reaction mixture was stirred at 25° C. for one day. Aqueous 1M HCl solution was added thereto, and the resultant mixture was stirred at ambient temperature. After quenching the reaction, the mixture was extracted with ethyl acetate, and the extract was dried under reduced pressure. Recrystallization from methylene chloride and hexane gave Compound (17).

### Preparation of Compound (18)

In toluene, dissolved were 2-chloro-9,10-anthraquinone (18.0 mmol), Compound (19) (21.5 mmol), tetrakis palladium (0) triphenylphosphine (Pd(PPh₃)₄) (2.2 mmol) and Aliquat 336 (3.0 mmol). Aqueous 2M potassium carbonate solution was added to the solution, and the mixture was stirred under reflux for 3 hours. Then the temperature was lowered to 25° C., and distilled water was added thereto to quench the reaction. The reaction mixture was extracted with ethyl acetate, and the extract was dried under reduced pressure. Recrystallization from methanol and tetrahydrofuran gave Compound (18).

### Preparation of Compound (21)

To the bromo compound (Compound 19 or 20) (30.3 mmol), tetrahydrofuran was added, and the mixture was stirred at 25° C. for 10 minutes to achieve complete dissolution. The temperature was then lowered to −72° C., and n-butyl lithium (2.5 M in hexane) (36.3 mmol) was slowly added dropwise thereto. After 1 hour, Compound (18) (12.1 mmol) was added, and the temperature was slowly raised to 25° C. After stirring the mixture for 26 hours at the same temperature, saturated aqueous ammonium chloride solution was added, and the resultant mixture was stirred for 1 hour. The organic layer separated after filtration under reduced pressure was evaporated to obtain Compound (21).

### Preparation of Compound (1)

Compound (21) (9.9 mmol) obtained as above, potassium iodide (KI) (39.6 mmol) and sodium phosphate monohydrate (NaH₂PO₂.H₂O) (59.3 mmol) were dissolved in acetic acid, and the solution was stirred under reflux for 21 hours. After cooling to 25° C., water was added to the reaction mixture, and the resultant mixture was stirred. Solid produced was filtered and washed sequentially with methanol, ethyl acetate and tetrahydrofuran to obtain the target compound (1) as light ivory product.

### Preparation Example 1

### Preparation of Compound (101)

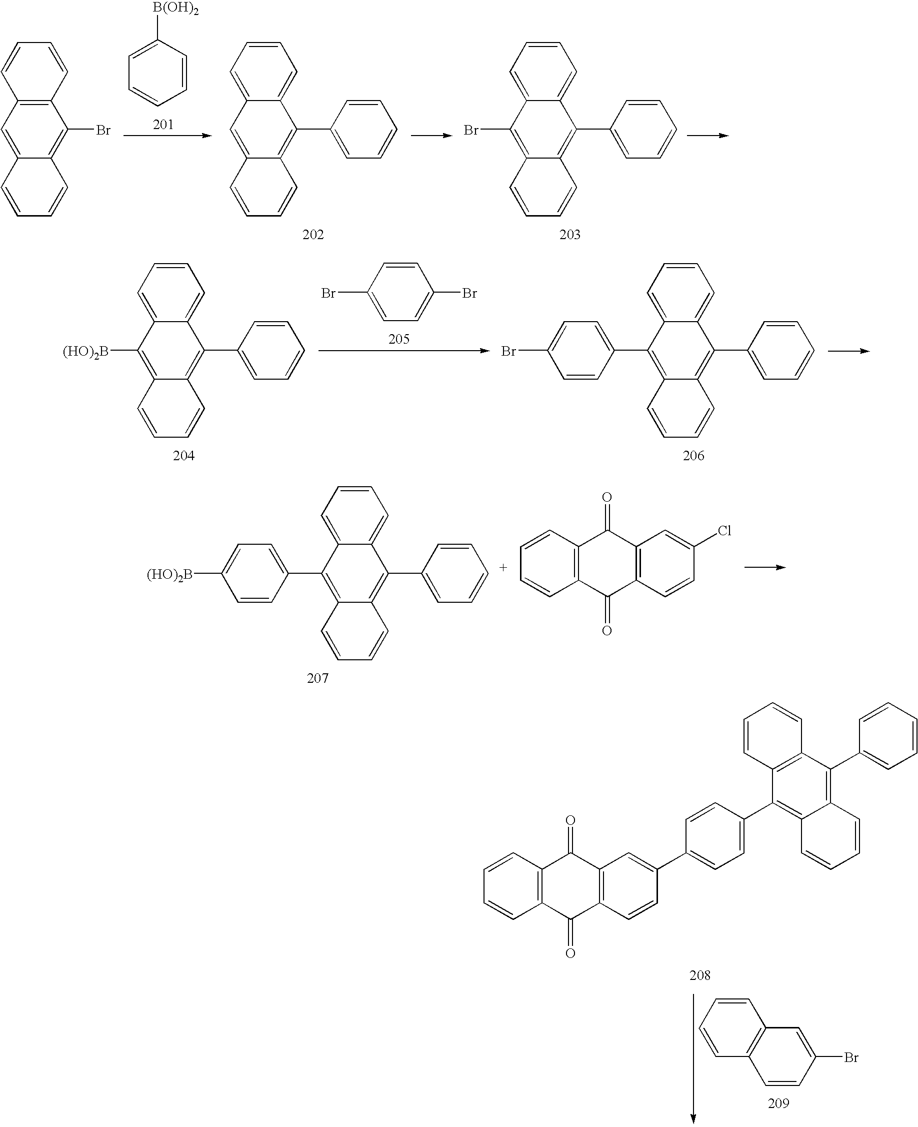

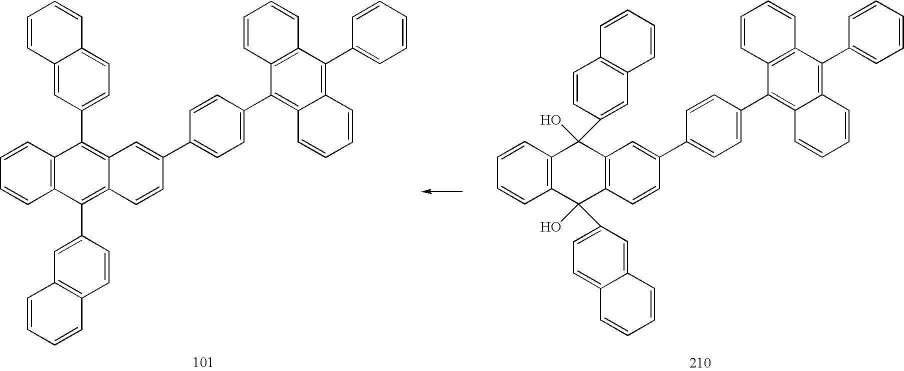

### Preparation of Compound (202)

In a mixed solution of toluene (300 mL) and ethanol (150 mL), dissolved were 9-bromoanthracene (15.0 g, 58.3 mmol), phenylboronic acid (Compound 201) (8.5 g, 70.0 mmol) and tetrakis palladium (0) triphenylphosphine (Pd(PPh₃)₄) (6.7 g, 5.8 mmol). After adding aqueous 2M sodium carbonate solution (145 mL) thereto, the resultant mixture was stirred under reflux at 120° C. for 5 hours. Then, the temperature was lowered to 25° C., and the reaction was quenched by adding distilled water (150 mL). The reaction mixture was extracted with ethyl acetate (200 mL), and the extract was dried under reduced pressure. Recrystallization from tetrahydrofuran (10 mL) and methanol (300 mL) gave Compound (202) (12.0 g, 47.2 mmol, 81.0%).

### Preparation of Compound (203)

Under nitrogen atmosphere, Compound (202) (11.7 g, 46.0 mmol) and N-bromosuccinimide (9.0 g, 50.6 mmol) were dissolved in dichloromethane (360 mL). The resultant solution was then stirred at 25° C. for 5 hours. The reaction was quenched by adding distilled water (300 mL), and the reaction mixture was extracted with dichloromethane (200 mL). The extract was dried under reduced pressure, and recrystallized from tetrahydrofuran (20 mL) and methanol (200 mL) to obtain the target compound (203) (13.0 g, 39.0 mmol, 84.8%).

### Preparation of Compound (204)

In thoroughly purified tetrahydrofuran (200 mL), Compound (203) (13.0 g, 39.0 mmol) was dissolved. The resultant solution was chilled to −78° C., and n-butyl lithium (1.6 M in hexane) (29.3 mL, 46.8 mmol) was slowly added thereto. After stirring the mixture for 1 hour, added was trimethyl borate (8.7 mL, 78.0 mmol). The temperature was slowly raised to 25° C., and the mixture was stirred at the same temperature for one day. Aqueous 1 M HCl solution (200 mL) was added thereto, and the mixture was stirred at ambient temperature for 5 hours. The reaction was quenched, and the reaction mixture was extracted with ethyl acetate (300 mL) and the extract was dried under reduced pressure. Recrystallization from methylene chloride (20 mL) and hexane (200 mL) gave the target compound (204) (9.6 g, 32.1 mmol, 82.3%).

### Preparation of Compound (206)

In a mixed solution of toluene (300 mL) and ethanol (150 mL), dissolved were 1,4-dibromobenzene (7.6 g, 32.1 mmol), Compound (204) (9.6 g, 32.1 mmol) and tetrakis palladium (0) triphenylphosphine (Pd(PPh₃)₄) (3.7 g, 3.2 mmol). After adding aqueous 2M sodium carbonate solution (145 mL) thereto, the resultant mixture was stirred under reflux at 120° C. for 5 hours. Then, the temperature was lowered to 25° C., and the reaction was quenched by adding distilled water (150 mL). The mixture was extracted with ethyl acetate (200 mL), and the extract was dried under reduced pressure. Recrystallization from tetrahydrofuran (20 mL) and methanol (300 mL) gave the target compound (206) (10.7 g, 26.1 mmol, 81.3%).

### Preparation of Compound (207)

In thoroughly purified tetrahydrofuran (200 mL), Compound (206) (10.7 g, 26.1 mmol) was dissolved. The resultant solution was chilled to −78° C., and n-butyl lithium (1.6 M in hexane) (19.6 mL, 31.3 mmol) was slowly added thereto. After stirring the mixture for 1 hour, added was trimethyl borate (3.50 mL, 31.3 mmol). The temperature was slowly raised to 25° C., and the mixture was stirred at the same temperature for one day. Aqueous 1 M HCl solution (200 mL) was added thereto, and the mixture was stirred at ambient temperature for 5 hours. The reaction was quenched, and the reaction mixture was extracted with ethyl acetate (300 mL). The extract was dried under reduced pressure. Recrystallization from methylene chloride (20 mL) and hexane (200 mL) gave the target compound (207) (8.04 g, 21.5 mmol, 82.4%).

### Preparation of Compound (208)

In toluene (300 mL), dissolved were 2-chloro-9,10-anthraquinone (3.7 g, 18.0 mmol), Compound (207) (8.0 g, 21.5 mmol), tetrakis palladium (0) triphenylphosphine (Pd(PPh₃)₄) (2.5 g, 2.2 mmol) and Aliquat 336 (1.4 mL, 3.0 mmol). After adding aqueous 2M potassium carbonate solution (150 mL) thereto, the resultant mixture was stirred under reflux for 3 hours. Then, the temperature was lowered to 25° C., and the reaction was quenched by adding distilled water (100 mL). The mixture was extracted with ethyl acetate (200 mL), and the extract was dried under reduced pressure. Recrystallization from methanol (200 mL) and tetrahydrofuran (50 mL) gave the target compound (208) (6.5 g, 12.1 mmol, 67.2%).

### Preparation of Compound (210)

Tetrahydrofuran (250 mL) was added to 2-bromonaphthalene (Compound 209) (6.3 g, 30.3 mmol), and the mixture was stirred at 25° C. for 10 minutes to achieve complete dissolution. After chilling to −72° C., n-butyl lithium (2.5 M in hexane) (14.5 mL, 36.3 mmol) was slowly added dropwise. After 1 hour, Compound (208) (6.5 g, 12.1 mmol) was added thereto, and the temperature was slowly raised to 25° C. After stirring the reaction mixture for 26 hours, saturated aqueous ammonium chloride solution was added thereto, and the resultant mixture was stirred for 1 hour. Filtration under reduced pressure, separation of organic layer, and evaporation gave the target compound (210) (7.8 g, 9.9 mmol, 81.7%).

### Preparation of Compound (101)

Compound (210) (7.8 g, 9.9 mmol), potassium iodide (KI) (6.6 g, 39.6 mmol) and sodium phosphate monohydrate (NaH₂PO₂.H₂O) (6.3 g, 59.3 mmol) were dissolved in acetic acid (150 mL), and the solution was stirred under reflux for 21 hours. After cooling the solution to 25° C., water (200 mL) was added with stirring, and the solid generated was filtered. The solid obtained was washed sequentially with methanol (300 mL), ethyl acetate (100 mL) and tetrahydrofuran (50 mL), to provide the target compound (101) (5.3 g, 7.0 mmol, 71.2%) as light ivory product.

¹H NMR (CDCl₃, 300 MHz) δ=7.23 (m, 3H), 7.32-7.35 (m, 12H), 7.48-7.54 (m, 5H), 7.67-7.73 (m, 13H), 7.89-7.93 (m, 5H).

MS/FAB: 759.2 (found) 758.2 (calculated for C₆₀H₃₈)

### Preparation Example 2-36

The organic electroluminescent compounds listed in Table were prepared according to the procedures described in Preparation Example 1, and the ¹H NMR and MS/FAB data of the compounds are shown in Table 2.

TABLE 1

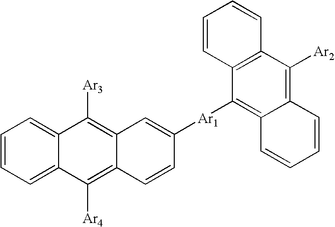

| Preparation Ex. | Compound No. | Ar₁ | Ar₂ | Ar₃ | Ar₄ |
|---|---|---|---|---|---|
| 1 | 101 | 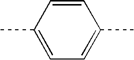 | 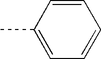 | 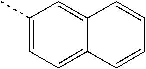 | 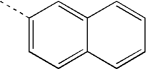 |
| 2 | 102 | 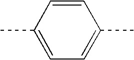 | 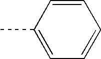 | 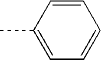 | 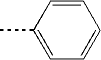 |
| 3 | 103 | 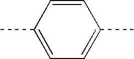 | 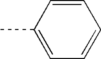 | 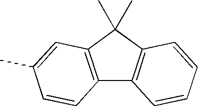 | 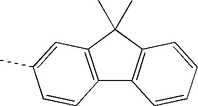 |
| 4 | 104 | 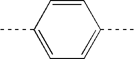 | 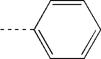 | 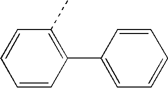 | 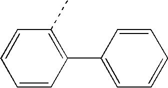 |
| 5 | 105 | 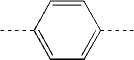 | 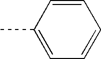 | 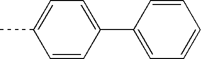 | 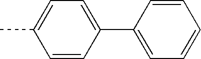 |
| 6 | 106 | 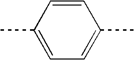 | 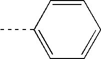 | 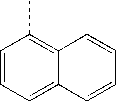 | 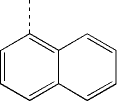 |
| 7 | 107 | 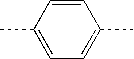 | 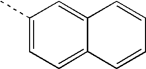 | 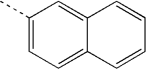 | 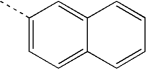 |
| 8 | 108 | 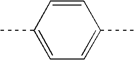 | 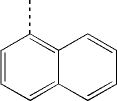 | 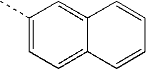 | 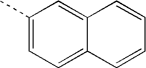 |
| 9 | 109 | 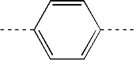 | 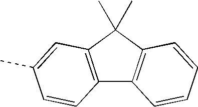 | 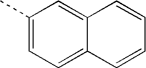 | 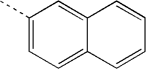 |
| 10 | 110 | 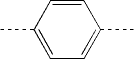 | 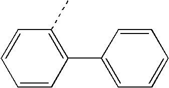 | 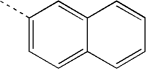 | 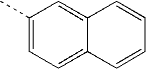 |
| 11 | 111 | 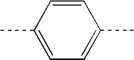 | 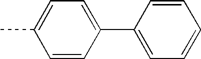 | 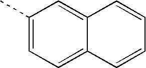 | 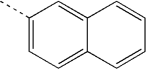 |
| 12 | 112 | 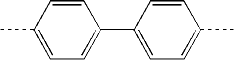 | 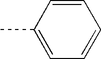 | 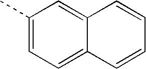 | 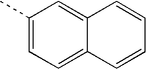 |
| 13 | 113 | 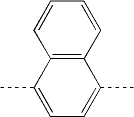 | 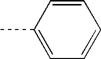 | 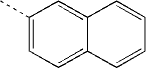 | 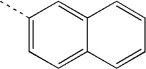 |
| 14 | 114 | 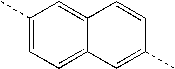 | 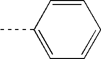 | 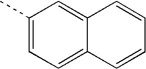 | 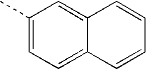 |
| 15 | 115 | 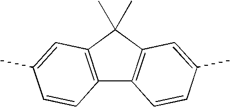 | 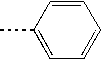 | 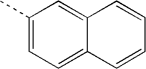 | 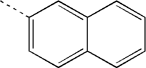 |
| 16 | 116 | 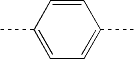 | 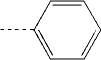 | 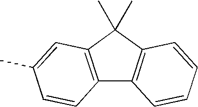 | 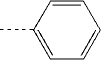 |
| 17 | 117 | 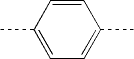 | 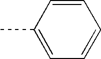 | 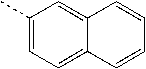 | 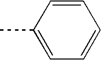 |
| 18 | 118 | 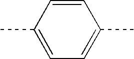 | 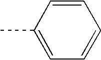 | 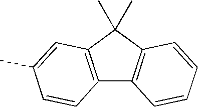 | 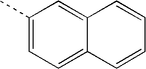 |
| 19 | 119 | 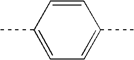 | 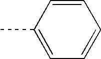 | 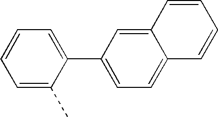 | 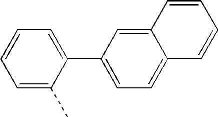 |
| 20 | 120 | 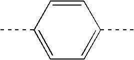 | 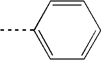 | 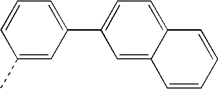 | 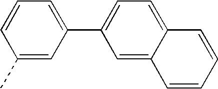 |
| 21 | 121 | 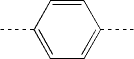 | 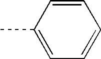 | 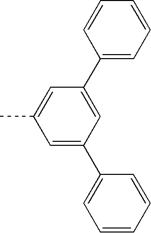 | 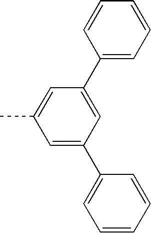 |
| 22 | 122 | 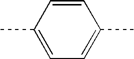 | 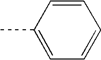 | 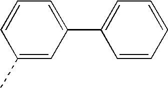 | 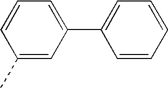 |
| 23 | 123 | 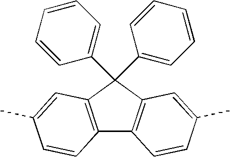 | 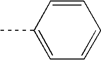 | 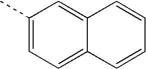 | 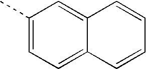 |
| 24 | 124 | 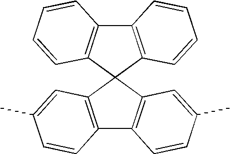 | 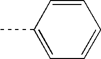 | 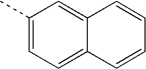 | 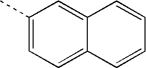 |
| 25 | 125 | 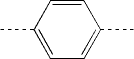 | 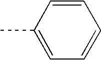 | 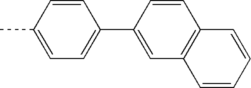 | 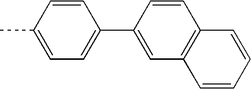 |
| 26 | 126 | 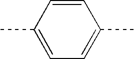 | 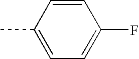 | 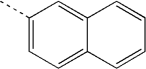 | 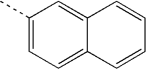 |
| 27 | 127 | 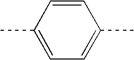 | 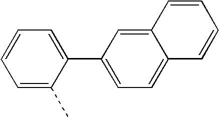 | 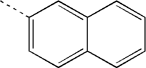 | 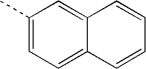 |
| 28 | 128 | 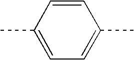 | 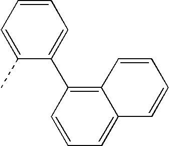 | 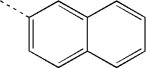 | 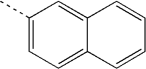 |
| 29 | 129 | 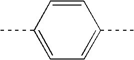 | 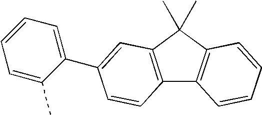 | 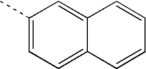 | 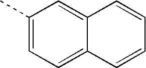 |
| 30 | 130 | 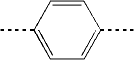 | 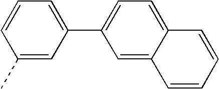 | 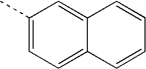 | 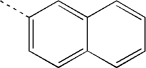 |
| 31 | 131 | 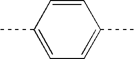 | 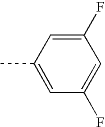 | 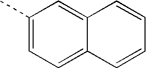 | 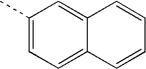 |
| 32 | 132 | 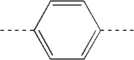 | 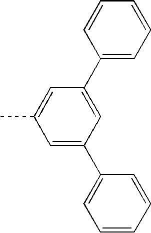 | 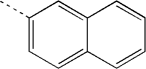 | 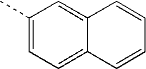 |
| 33 | 133 | 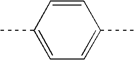 | 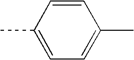 | 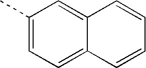 | 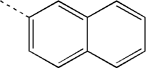 |
| 34 | 134 | 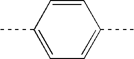 | 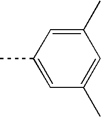 | 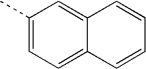 | 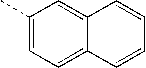 |
| 35 | 135 | 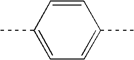 | 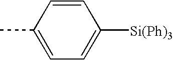 | 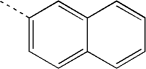 | 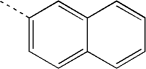 |
| 36 | 136 | 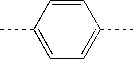 | 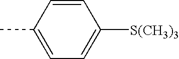 | 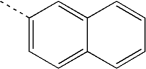 | 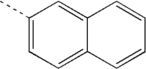 |

TABLE 2

| Compound | | MS/FAB | |
|---|---|---|---|
| No. | ¹H NMR (CDCl₃, 300 MHz) | found | calculated |
| 101 | δ = 7.23(m, 3H), 7.32-7.35(m, 12H), 7.48-7.54(m, 5H), | 759.2 | 758.2 |
| | 7.67-7.73(m, 13H), 7.89-7.93 (m, 5H). | | |
| 102 | δ = 7.23(m, 2H), 7.32-7.35(m, 8H), 7.48-7.54(m, 6H), | 659.2 | 658.2 |
| | 7.67-7.73(m, 13H), 7.89-7.93 (m, 5H) | | |
| 103 | δ = 1.67 (s, 12H), 7.23(m, 4H), 7.32-7.35(m, 8H), | 891.3 | 890.3 |
| | 7.48-7.54(m, 8H), 7.67-7.73(m, 13H), 7.89-7.93 (m, 5H) | | |
| 104 | δ = 7.23(m, 6H), 7.32-7.35(m, 12H), 7.48-7.54(m, 6H), | 811.3 | 810.3 |
| | 7.67-7.73(m, 13H), 7.89-7.93 (m, 5H) | | |
| 105 | δ = 7.23(m, 2H), 7.32-7.35(m, 8H), 7.48-7.54(m, 10H), | 811.3 | 810.3 |
| | 7.67-7.73(m, 17H), 7.89-7.93 (m, 5H). | | |
| 106 | δ = 7.23(m, 2H), 7.32-7.35(m, 12H), 7.48-7.54(m, 6H), | 759.2 | 758.2 |
| | 7.67-7.73(m, 13H), 7.89-7.93 (m, 5H). | | |
| 107 | δ = 7.23(m, 3H), 7.32-7.35(m, 14H), 7.48-7.54(m, 5H), | 809.3 | 808.3 |
| | 7.67-7.73(m, 13H), 7.89-7.93 (m, 5H). | | |
| 108 | δ = 7.22(m, 2H), 7.34(m, 14H), 7.49(m, 6H), | 809.3 | 808.3 |
| | 7.71(m, 13H), 7.91(m, 5H). | | |
| 109 | δ = 1.71(s, 6H), 7.21(m, 2H), 7.37(m, 12H), | 875.3 | 874.3 |
| | 7.48(m, 8H), 7.71(m, 11H), 7.91(m, 7H). | | |
| 110 | δ = 7.22(m, 4H), 7.39(m, 12H), 7.51(m, 8H), | 835.3 | 834.3 |
| | 7.69(m, 11H), 7.88(m, 7H). | | |
| 111 | δ = 7.22(m, 2H), 7.39(m, 10H), 7.51(m, 8H), | 835.3 | 834.3 |
| | 7.62(m, 11H), 7.73(m, 4H), 7.88(m, 7H). | | |
| 112 | δ = 7.23(m, 3H), 7.32-7.35(m, 10H), 7.48-7.54(m, 11H), | 835.3 | 834.3 |
| | 7.67-7.73(m, 13H), 7.89-7.93 (m, 5H). | | |
| 113 | δ = 7.23(m, 3H), 7.37(m, 10H), 7.51(m, 9H), | 809.3 | 808.3 |
| | 7.69(m, 13H), 7.88(m, 5H). | | |
| 114 | δ = 7.22(m, 2H), 7.43(m, 11H), 7.56(m, 7H), | 809.3 | 808.3 |
| | 7.71(m, 15H), 7.93(m, 5H). | | |
| 115 | δ = 1.72(s, 6H), 7.24(m, 2H), 7.42~7.45(m, 9H), | 8754.3 | 874.3 |
| | 7.53~7.57(m, 9H), 7.70-7.74(m, 11H), 7.85(m, 4H), | | |
| | 7.94(m, 5H). | | |
| 116 | δ = 1.68(s, 6H), 7.22~7.38(m, 14H), 7.48~7.55(m, 10H), | 774.3 | 774.9 |
| | 7.60~7.67(m, 7H), 7.73~7.77(m, 2H), 7.84~7.90(m, 3H). | | |
| 117 | δ = 7.22(m, 2H), 7.32(m, 12H), 7.48~7.54(m, 10H), | 708.3 | 708.9 |
| | 7.67(m, 8H), 7.73(m, 2H), 7.89(m, 2H). | | |
| 118 | δ = 1.67(s, 6H), 7.22~7.28(m, 2H), 7.32~7.38(m, 11H), | 824.3 | 825.0 |
| | 7.48~7.55(m, 9H), 7.60~7.67(m, 9H), 7.73~7.77(m, 3H), | | |
| | 7.84~7.90(m, 4H). | | |
| 119 | δ = 7.22~7.28(m, 5H), 7.32(m, 12H), 7.48~7.54(m, 13H), | 910.3 | 911.4 |
| | 7.67(m, 10H), 7.73(m, 3H), 7.89(m, 3H). | | |
| 120 | δ = 7.22(m, 2H), 7.32~7.38(m, 14H), 7.44~7.48(m, 5H), | 910.3 | 911.4 |
| | 7.54(m, 7H), 7.67(m, 10H), 7.73~7.70(m, 5H), 7.89(m, 3H). | | |
| 121 | δ = 7.22(m, 5H), 7.32(m, 16H), 7.48(m, 10H), 7.54(m, 5H), | 962.4 | 963.2 |
| | 7.66~7.67(m, 12H), 7.73(m, 1H), 7.89(m, 1H). | | |
| 122 | δ = 7.22(m, 3H), 7.32~7.38(m, 14H), 7.44~7.48(m, 10H), | 810.3 | 811.0 |
| | 7.54(m, 5H), 7.67(m, 6H), 7.70~7.73(m, 3H), 7.89(m, 1H). | | |
| 123 | δ = 7.06~7.07(m, 6H), 7.14(m, 4H), 7.22(m, 1H), | 998.4 | 999.2 |
| | 7.32(m, 12H), 7.48~7.54(m, 5H), 7.60~7.67(m, 12H), | | |
| | 7.73~7.77(m, 5H), 7.89~7.90(m, 5H). | | |
| 124 | δ = 7.16~7.19(m, 4H), 7.22(m, 1H), 7.32~7.35(m, | 996.4 | 997.2 |
| | 14H), 7.48~7.54(m, 5H), 7.60~7.67(m, 12H), | | |
| | 7.72~7.77(m, 7H), 7.89~7.90(m, 5H). | | |
| 125 | δ = 7.22(m, 1H), 7.32(m, 12H), 7.48(m, 2H), 7.54(m, 15H), | 910.4 | 911.1 |
| | 7.67(m, 10H), 7.73(m, 3H), 7.89(m, 3H). | | |
| 126 | δ = 7.03(m, 2H), 7.32(m, 10H), 7.46(m, 2H), 7.54(m, 7H), | 776.3 | 776.9 |
| | 7.67(m, 10H), 7.73(m, 3H), 7.89(m, 3H). | | |
| 127 | δ = 7.28~7.32(m, 14H), 7.54(m, 10H), 7.67(m, 12H), | 884.3 | 885.1 |
| | 7.73(m, 4H), 7.89(m, 4H). | | |
| 128 | δ = 7.28(m, 2H), 7.32~7.38(m, 13H), 7.54(m, 10H), | 884.3 | 885.1 |
| | 7.03~7.67(m, 13H), 7.73(m, 3H), 7.89(m, 3H). | | |
| 129 | δ = 1.68(s, 6H), 7.28(m, 3H), 7.32~7.38(m, 11H), | 950.4 | 951.2 |
| | 7.54~7.55(m, 10H), 7.60~7.67(m, 11H), 7.73~7.77(m, 4H), | | |
| | 7.84~7.90(m, 5H). | | |
| 130 | δ = 7.32~7.38(m, 13H), 7.44(m, 2H), 7.54(m, 8H), | 884.3 | 885.1 |
| | 7.67(m, 12H), 7.70~7.73(m, 5H), 7.89(m, 4H). | | |
| 131 | δ = 6.64(t, 1H), 6.96(m, 2H), 7.32(m, 10H), 7.54(m, 7H), | 794.2 | 794.9 |
| | 7.67(m, 10H), 7.73(m, 3H), 7.89(m, 3H). | | |
| 132 | δ = 7.22(m, 2H), 7.32(m, 14H), 7.48(m, 4H), 7.54(m, 7H), | 910.4 | 911.1 |
| | 7.66~7.67(m, 13H), 7.73(m, 3H), 7.89(m, 3H). | | |
| 133 | δ = 2.35(s, 3H), 7.12(m, 2H), 7.32~7.36(m, 12H), | 772.3 | 773.0 |
| | 7.54(m, 7H), 7.67(m, 10H), 7.73(m, 3H), 7.89(m, 3H). | | |
| 134 | δ = 2.35(s, 6H), 6.82(s, 1H), 7.09(d, 2H), 7.32(m, 10H), | 786.3 | 787.0 |
| | 7.54(m, 7H), 7.67(m, 10H), 7.73(m, 3H), 7.89(m, 3H). | | |
| 135 | δ = 7.32~7.36(m, 19H), 7.54~7.58(m, 15H), | 1016.4 | 1017.3 |
| | 7.60~7.67(m, 12H), 7.73(m, 3H), 7.89(d, 3H). | | |
| 136 | δ = 0.66(s, 9H), 7.32(m, 10H), 7.46(m, 2H), 7.54(m, 9H), | 830.3 | 831.1 |
| | 7.60~7.67(m, 10H), 7.73(m, 3H), 7.89(d, 3H). | | |

### Examples 1-7

### Manufacture of OLED's Using the Compounds According to the Invention

OLED's were manufactured by using the electroluminescent materials according to the invention.

First, a transparent electrode ITO thin film (2) (15Ω/□) obtained from glass (1) for OLED was subjected to ultrasonic washing with trichloroethylene, acetone, ethanol and distilled water, sequentially, and stored in isopropanol before use.

Then, an ITO substrate was equipped in a substrate folder of a vacuum vapor-deposit device, and 4,4′,4″-tris(N,N-(2-naphthyl)-phenylamino)triphenylamine (2-TNATA) was placed in a cell of the vacuum vapor-deposit device, which was then vented to reach 10⁻⁶ torr of vacuum in the chamber. Electric current was applied to the cell to evaporate 2-TNATA to vapor-deposit a hole injection layer (3) with a thickness of 60 nm on the ITO substrate.

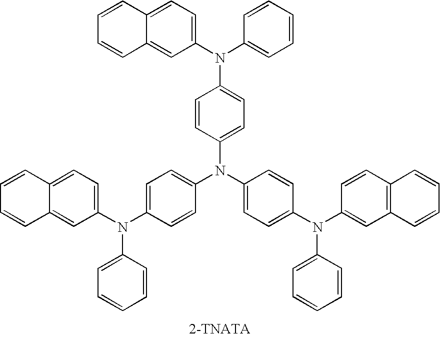

Then, another cell of the vacuum vapor-deposit device was charged with N,N′-bis(α-naphthyl)-N,N′-diphenyl-4,4′-diamine (NPB), and electric current was applied to the cell to evaporate NPB to vapor-deposit a hole transportation layer (4) with a thickness of 20 nm on the hole injection layer.

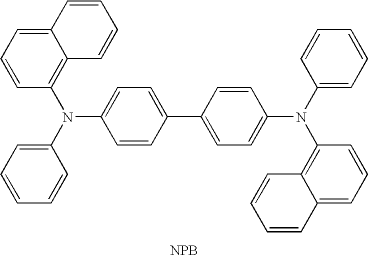

After formation of the hole injection layer and the hole transportation layer, an electroluminescent layer was vapor-deposited as follows. One cell of the vacuum vapor-deposit device was charged with a compound according to the present invention (for example, Compound 121), while another cell of said device was charged with perylene (having the structure shown below) as a dopant material. Two substances were doped by evaporating with different rates to vapor-deposit an electroluminescent layer (5) with a thickness of 35 nm on the hole transportation layer, in a doping concentration of 1 to 2 mol % of perylene.

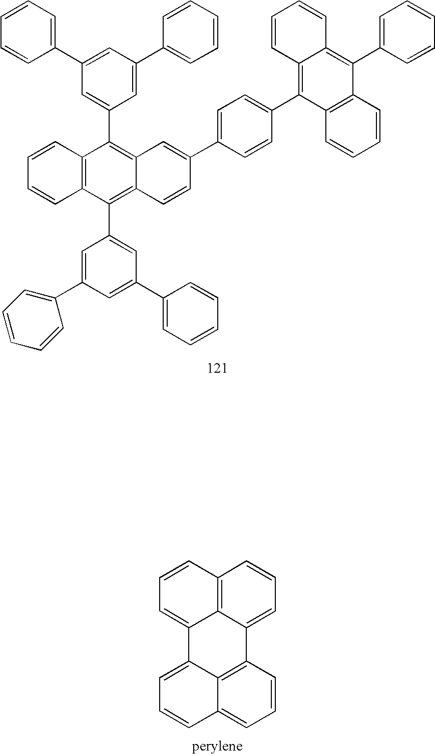

Then, tris(8-hydroxyquinoline)-aluminum (III) (Alq) having the structure shown below was vapor-deposited with a thickness of 20 nm as an electron transportation layer (6), followed by lithium quinolate (Liq) having the structure shown below with a thickness of 1˜2 nm as an electron injection layer (7). An Al cathode (8) was vapor-deposited by using another vacuum vapor-deposit device with a thickness of 150 nm, to manufacture an OLED.

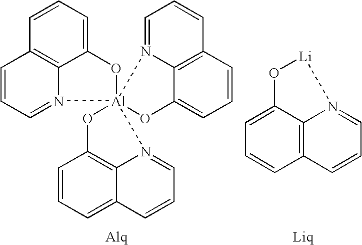

Each compound employed for an OLED was purified by vacuum sublimation under 10⁻⁶ torr, and employed as an electroluminescent material for an OLED.

### Examples 8-14

### Manufacture of OLED's Using the Compounds According to the Invention

After formation of a hole injection layer and a hole transportation layer as described in Example 1, an electroluminescent layer was vapor-deposited as follows. One cell of the vacuum vapor-deposit device was charged with a compound according to the invention (for example, Compound 121), while another cell of said device was charged with Coumarin 545T (C545T) having the structure shown below, respectively. The two substances were evaporated at different rates to carry out doping at a concentration of 1 to 2 mol % of Coumarin 545T (C545T) to provide an electroluminescent layer with a thickness of 35 nm on the hole transportation layer.

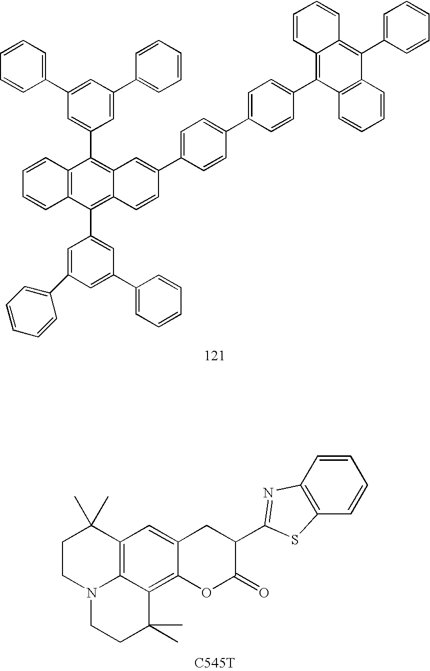

Then, an electron transportation layer and an electron injection layer were vapor-deposited according to the same procedure as described in Example 1, and an Al cathode was vapor-deposited with a thickness of 150 nm by using another vacuum vapor-deposit device to manufacture an OLED.

### Comparative Example 1

### Manufacture of an OLED Using a Conventional EL Material

After formation of a hole injection layer and a hole transportation layer as described in Example 1, one cell of the vacuum deposition device was charged with dinaphthylanthracene (DNA) as a blue electroluminescent material, and another cell was charged with perylene as a blue EL material. With a vapor-deposition rate of 100:1, an electroluminescent layer with a thickness of 35 nm was vapor-deposited on the hole transportation layer.

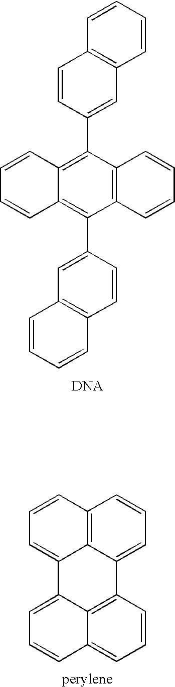

Then, an electron transportation layer and an electron injection layer were vapor-deposited according to the same procedure as described in Example 1, an Al cathode was vapor-deposited by using another vacuum vapor-deposit device with a thickness of 150 nm, to manufacture an OLED.

### Comparative Example 2

### Manufacture of an OLED Using a Conventional EL Material

After formation of a hole injection layer and a hole transportation layer as described in Example 1, another cell of the vacuum vapor-deposit device was charged with tris(8-hydroxyquinoline)aluminum (III) (Alq) as an electroluminescent host material, and still another cell was charged with Coumarin 545T (C545T). Two substances were doped by evaporation at different rates to vapor-deposit an electroluminescent layer with a thickness of 30 nm on the hole transportation layer. Preferable doping concentration is from 1 to 2 mol % on the basis of Alq.

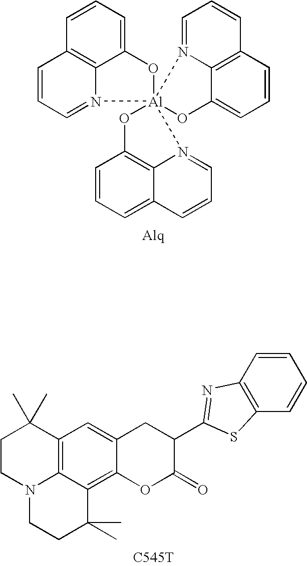

Then, an electron transportation layer and an electron injection layer were vapor-deposited according to the same procedure as described in Example 1, and an Al cathode was vapor-deposited by using another vacuum vapor-deposit device with a thickness of 150 nm, to manufacture an OLED.

### Comparative Example 3

### Manufacture of an OLED Using a Conventional EL Material

After formation of a hole injection layer and a hole transportation layer as described in Example 1, another cell of the vacuum deposition device was charged with dinaphthylanthracene (DNA) as a blue electroluminescent material, and still another cell was charged with Coumarin 545T (C545T) having the structure shown below. Two substances were doped by evaporation at different rates to vapor-deposit an electroluminescent layer with a thickness of 30 nm on the hole transportation layer. Preferable doping concentration is from 1 to 2 mol % on the basis of Alq.

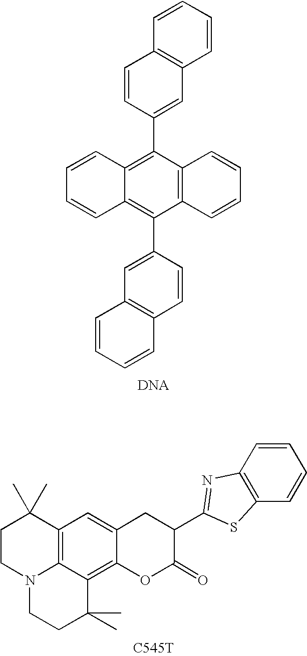

Then, an electron transportation layer and an electron injection layer were vapor-deposited according to the same procedure as described in Example 1, and an Al cathode was vapor-deposited by using another vacuum vapor-deposit device with a thickness of 150 nm, to manufacture an OLED.

### Comparative Example 4

### Manufacture of an OLED Using a Conventional EL Material

After formation of a hole injection layer and a hole transportation layer as described in Example 1, another cell of the vacuum deposition device was charged with Compound (A) from US Patent Publication No. 20060046097A1 as a blue electroluminescent material, and still another cell was charged with Coumarin 545T (C545T) having the structure shown below. Two substances were doped by evaporation at different rates to vapor-deposit an electroluminescent layer with a thickness of 30 nm on the hole transportation layer. Preferable doping concentration is from 1 to 2 mol % on the basis of Alq.

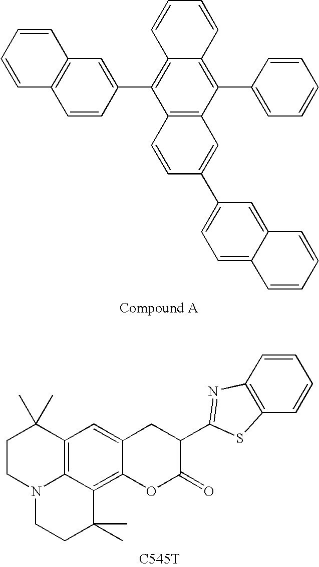

Then, an electron transportation layer and an electron injection layer were vapor-deposited according to the same procedure as described in Example 1, and an Al cathode was vapor-deposited by using another vacuum vapor-deposit device with a thickness of 150 nm, to manufacture an OLED.

### Experimental Example 1

### Blue and Green Electroluminescent Properties of OLED's Manufactured

Blue luminous efficiencies of OLED's comprising the organic electroluminescent compounds of the invention prepared from Examples 1-7 and a conventional electroluminescent (Comparative Example 1), and green luminous efficiencies of OLED's comprising the organic electroluminescent compounds of the invention prepared from Examples 8 to 14 and conventional electroluminescent compounds (Comparative Examples 2 to 4) were determined at 10,000 cd/m². The results are shown in Tables 3 and 4.

TABLE 3

| | | | | Luminous | |
|---|---|---|---|---|---|
| | | | Doping | efficiency (cd/ | |
| | | | conc. | A) @ 10,000 | EL |
| No. | Host | Dopant | (mol %) | cd/m² | color |
| Ex. 1 | Compound | Perylene | 2.0 | 4.5 | Blue |
| | 101 | | | | |
| Ex. 2 | Compound | Perylene | 2.0 | 4.8 | Blue |
| | 103 | | | | |
| Ex. 3 | Compound | Perylene | 2.0 | 4.7 | Blue |
| | 109 | | | | |
| Ex. 4 | Compound | Perylene | 2.0 | 4.2 | Blue |
| | 113 | | | | |
| Ex. 5 | Compound | Perylene | 2.0 | 4.9 | Blue |
| | 121 | | | | |
| Ex. 6 | Compound | Perylene | 2.0 | 4.2 | Blue |
| | 130 | | | | |
| Ex. 7 | Compound | Perylene | 2.0 | 4.6 | Blue |
| | 135 | | | | |
| Comp. Ex. 1 | DNA | Perylene | 2.0 | 2.4 | Blue |

TABLE 4

| | | | | Luminous | |
|---|---|---|---|---|---|
| | | | Doping | efficiency (cd/ | |
| | | | conc. | A) @ 10,000 | EL |
| No. | Host | Dopant | (mol %) | cd/m² | color |
| Ex. 8 | Compound | C545T | 2.0 | 17.8 | Green |
| | 101 | | | | |
| Ex. 9 | Compound | C545T | 2.0 | 18.2 | Green |
| | 103 | | | | |
| Ex. 10 | Compound | C545T | 2.0 | 18.4 | Green |
| | 109 | | | | |
| Ex. 11 | Compound | C545T | 2.0 | 18.1 | Green |
| | 113 | | | | |
| Ex. 12 | Compound | C545T | 2.0 | 19.8 | Green |
| | 121 | | | | |
| Ex. 13 | Compound | C545T | 2.0 | 18.6 | Green |
| | 130 | | | | |
| Ex. 14 | Compound | C545T | 2.0 | 17.8 | Green |
| | 135 | | | | |
| Comp. Ex. 2 | Alq | C545T | 2.0 | 8.0 | Green |
| Comp. Ex. 3 | DNA | C545T | 2.0 | 12.0 | Green |
| Comp. Ex. 4 | Compound | C545T | 2.0 | 12.7 | Green |
| | A | | | | |

Tables 3 and 4 exhibit the results of properties obtained when the electroluminescent materials of the present invention were applied to blue and green light-emitting devices. For both blue and green light-emitting devices, superior properties at high luminance could be commonly confirmed as compared to conventional electroluminescent materials.

The luminous efficiency was improved by 100% or more as compared to Alq host, and by 40% or more as compared to the conventional host in Comparative Example 3. These results elucidate definite overcome of limitation of conventional green electroluminescent materials. In particular, it is estimated that the excellent improvement of performance at high luminance sufficiently enables the compounds to be used in practical use for large screen OLED's, or manual OLED's of 2-inch level which require critical properties.

The EL material according to the present invention can be applied to both a blue OLED and a green OLED, and showed excellent results in view of performances. Those results show prominent characteristics as excellent EL materials. The invention of the material having those characteristics leads simplification of the structure of an OLED panel, to result in subsidiary result of reducing the production cost of an OLED. Due to the excellent features, innovative results may occur in development in the field of OLED's.

### INDUSTRIAL APPLICABILITY

The organic electroluminescent compounds according to the invention are advantageous in that they exhibit high luminous efficiency and excellent life property as an electroluminescent material, so that blue and green electroluminescent materials with very good operation lifetime of devices and organic light-emitting devices comprising the same can be obtained.

1. An organic electroluminescent material represented by Chemical Formula (1): 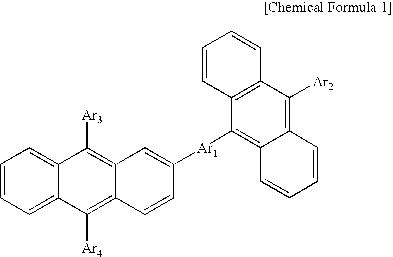
wherein, Ar₁ is a (C₅-C₂₀) aromatic ring or a fused polycyclic aromatic ring with two or more aromatic rings having been fused, provided that Ar₁ is not anthracenyl;
Ar₂ through Ar₄ independently represent a (C₅-C₂₀) aromatic ring or a fused polycyclic aromatic ring with two or more aromatic rings having been fused; and
the aromatic ring or the fused polycyclic aromatic ring with two or more aromatic rings having been fused of Ar₁ through Ar₄ may be further substituted by one or more substituent(s) selected from (C₁-C₂₀)alkyl, (C₁-C₂₀)alkoxy, halogen, tri(C₁-C₂₀)alkylsilyl, tri (C₆-C₂₀) arylsilyl, a (C₅-C₂₀) aromatic ring and a fused polycyclic aromatic ring with two or more aromatic rings having been fused.

2. An organic electroluminescent material according to claim 1, wherein
Ar₁ represents phenylene, biphenylene, naphthylene, spirobifluorenylene, phenanthrylene, triphenylenylene, pyrenylene, chrysenylene or naphthacenylene, and Ar₁ may be further substituted by (C₁-C₂₀)alkyl or phenyl; and
Ar₂ through Ar₄ independently represent phenyl, naphthyl, anthryl, biphenyl, fluorenyl, phenanthryl, triphenylenyl, pyrenyl, chrysenyl or naphthacenyl, and Ar₂ through Ar₄ may be further substituted by one or more substitutent(s) selected from (C₁-C₂₀)alkyl, (C₁-C₂₀)alkoxy, halogen, tri (C₁-C₂₀)alkylsilyl, tri (C₆-C₂₀)arylsilyl, phenyl, naphthyl, anthryl, fluorenyl, 9,9-dimethyl-fluoren-2-yl and 9,9-diphenyl-fluoren-2-yl.

3. An organic electroluminescent compound according to claim 2, which is selected from the compounds represented by one of the following chemical formulas:

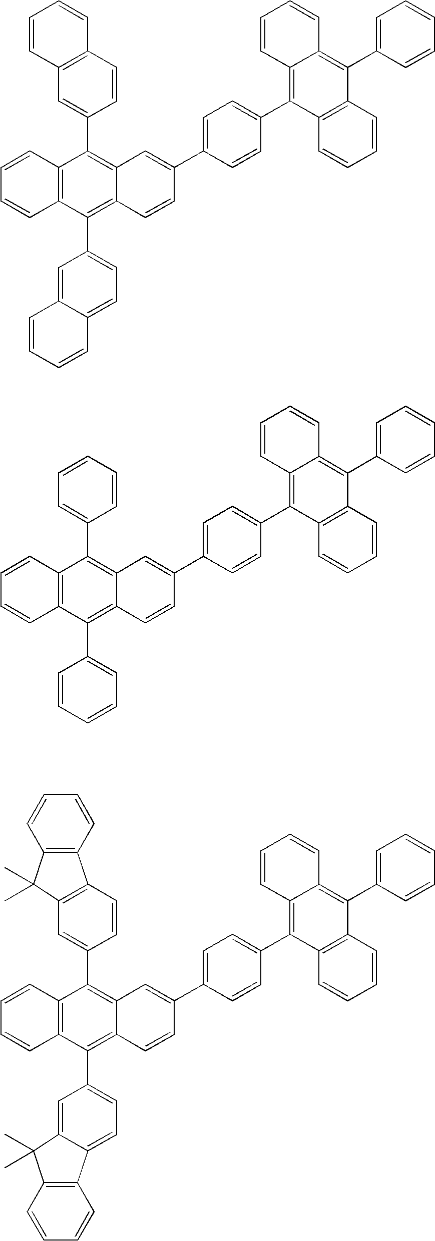

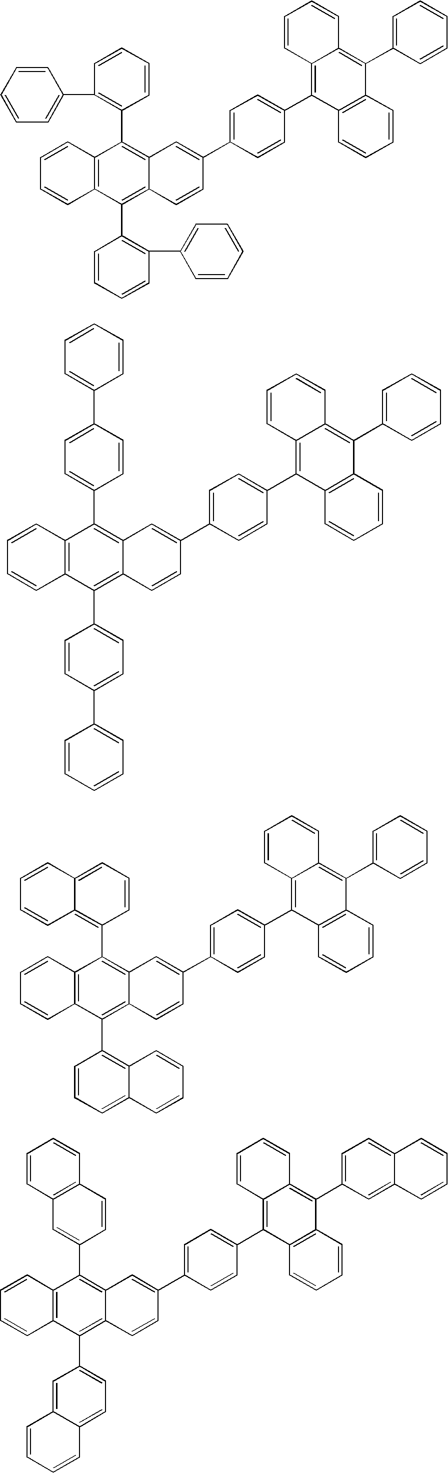

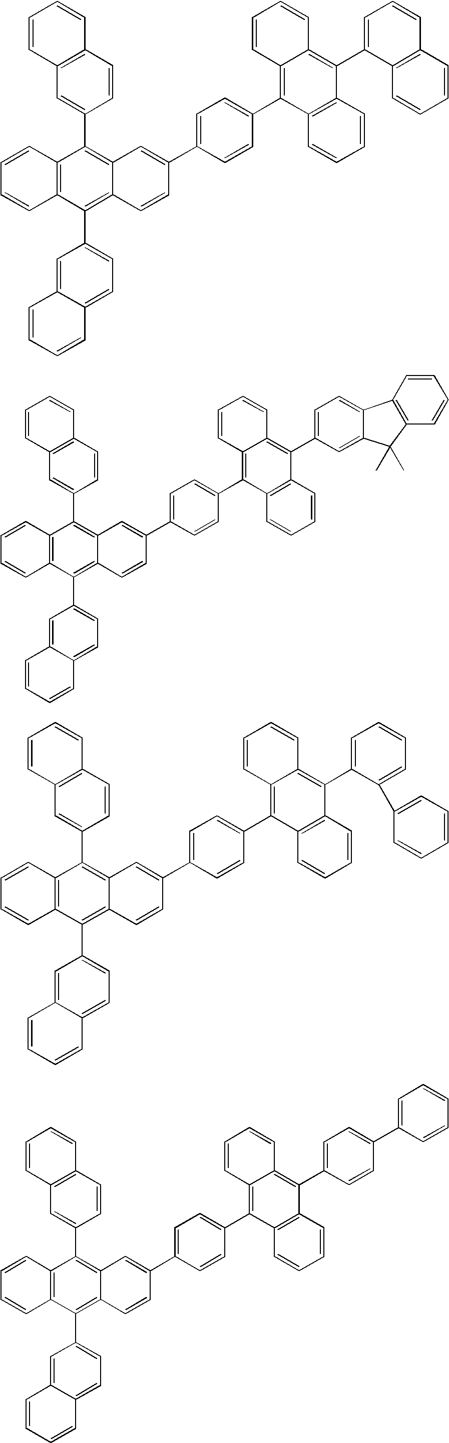

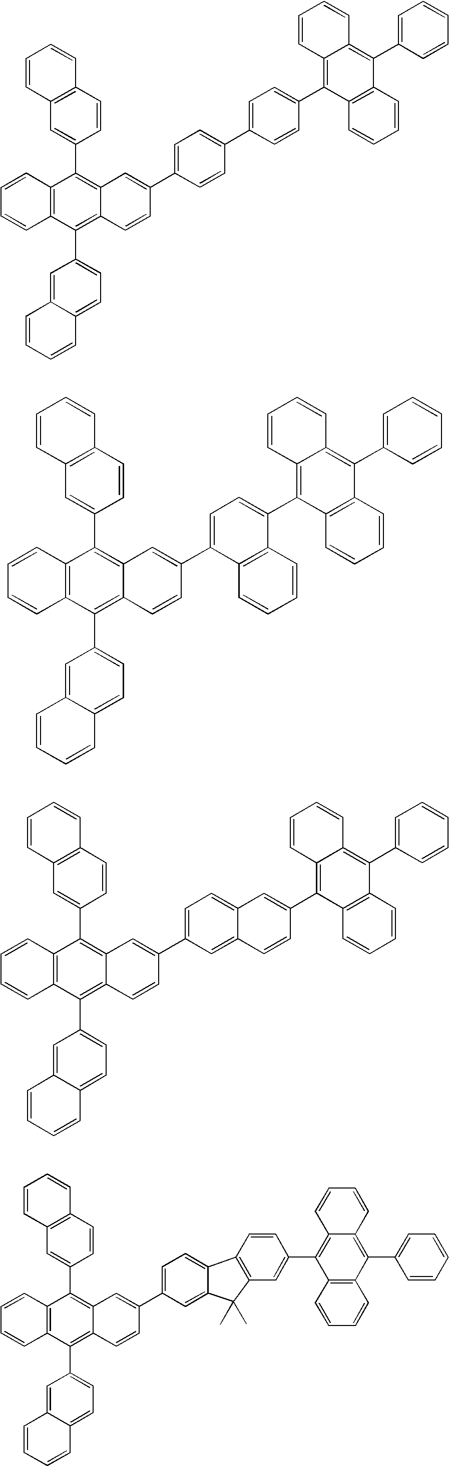

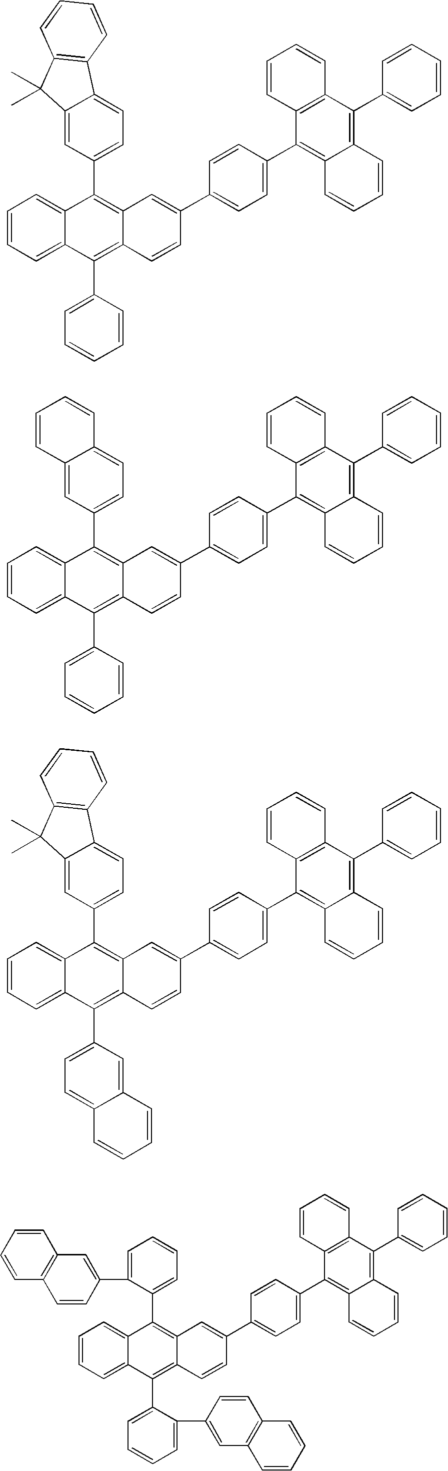

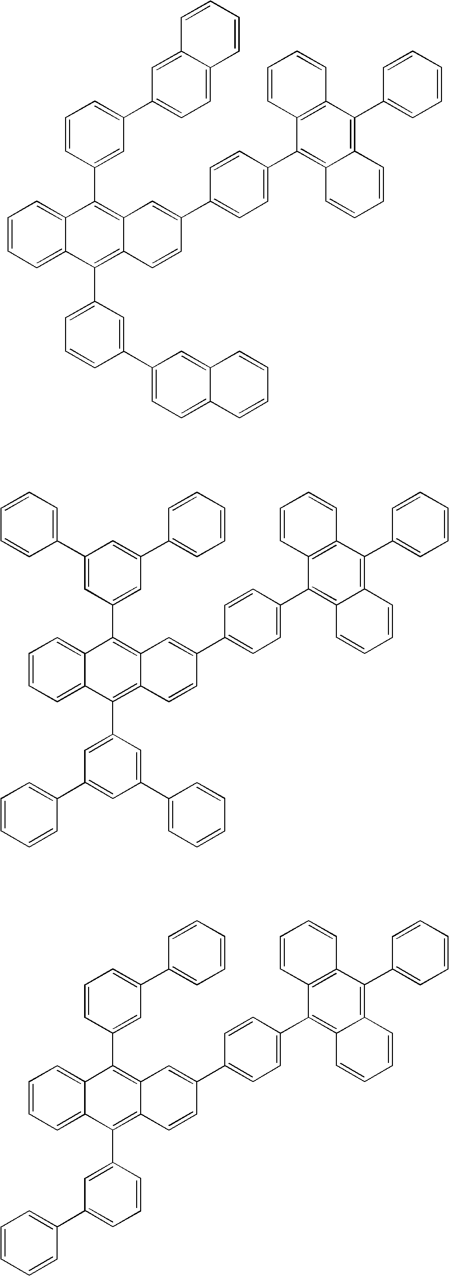

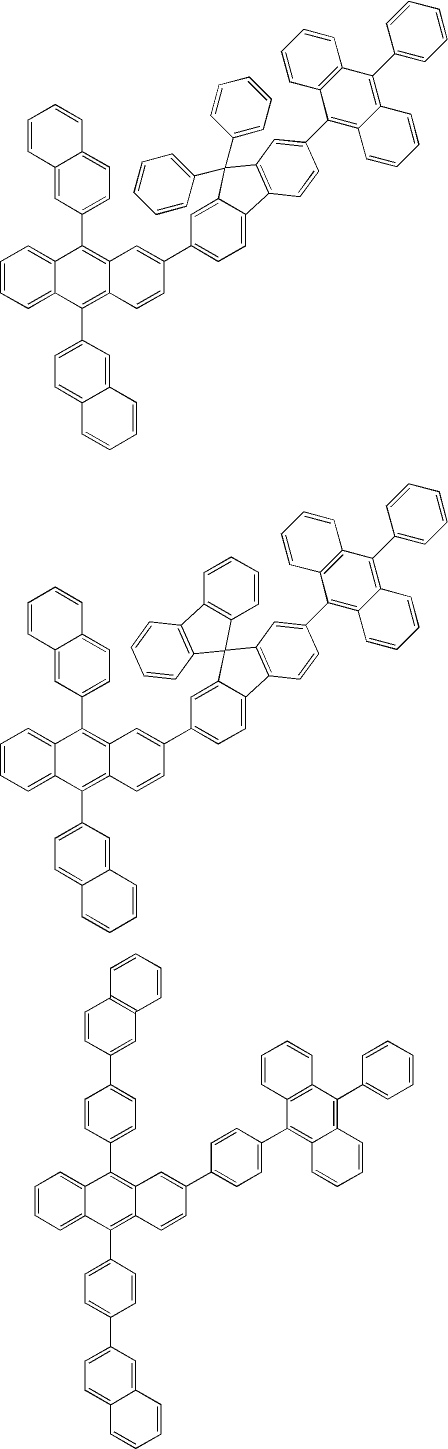

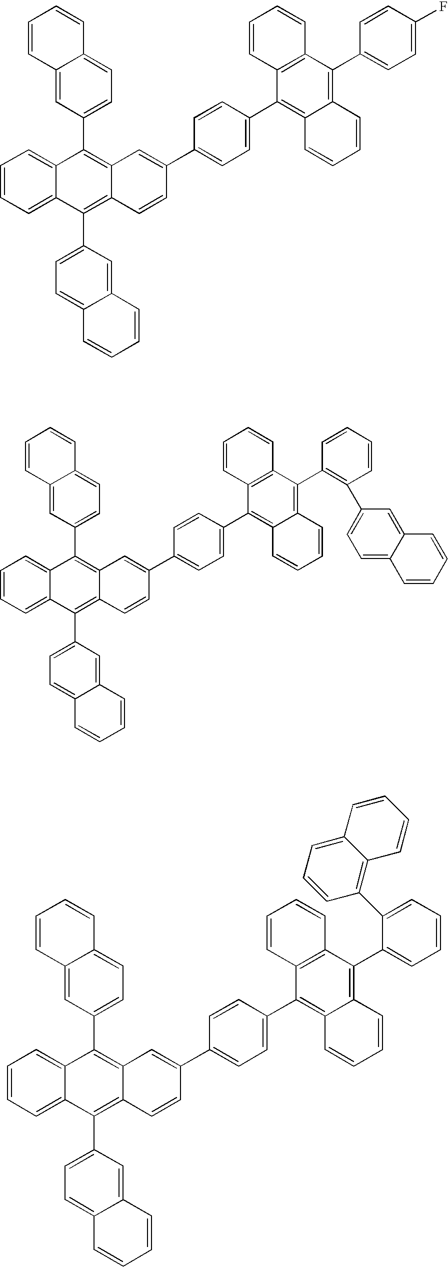

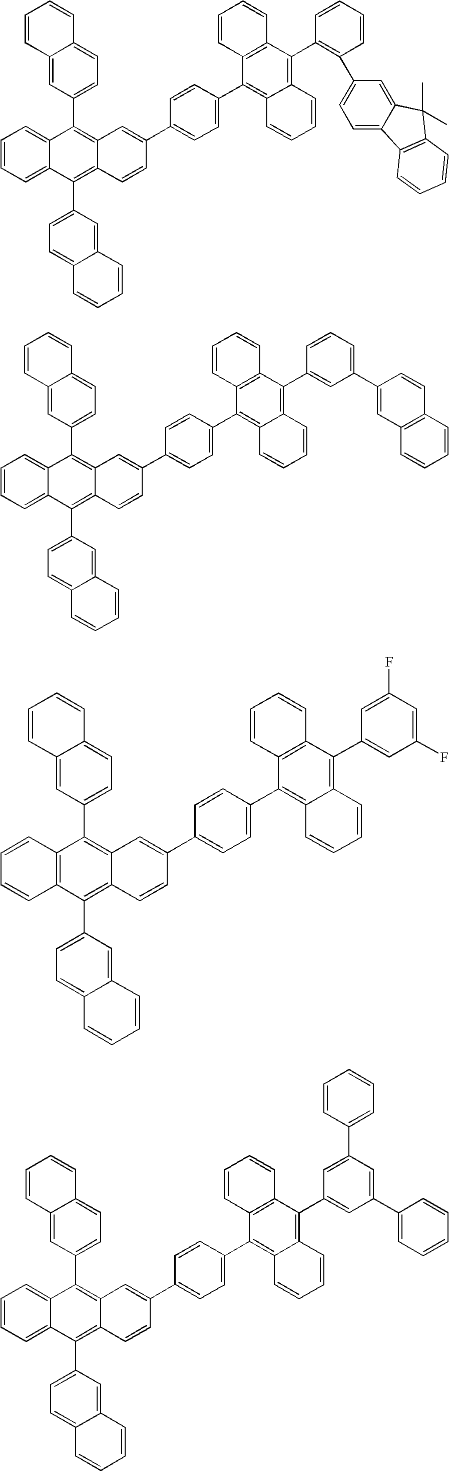

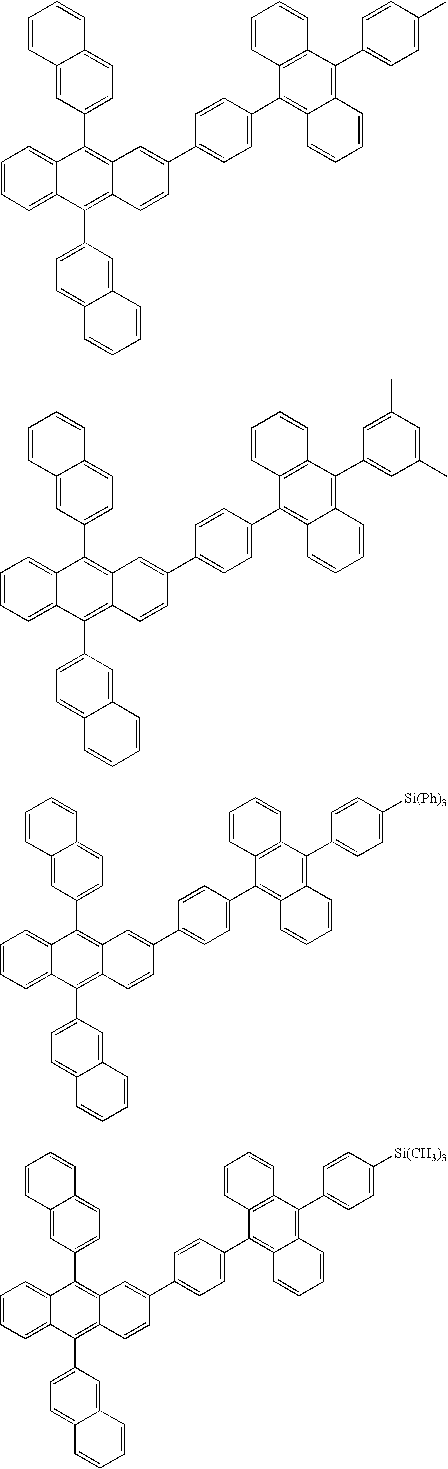

4. An organic light emitting device consisting of
a first electrode;
a second electrode; and
at least one organic layer(s) interposed between the first electrode and the second electrode;
wherein the organic layer comprises one or more organic compound(s) represented by Chemical Formula (1): 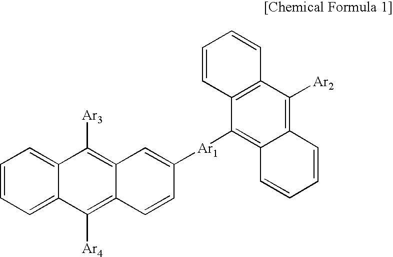
wherein, Ar₁ is a (C₅-C₂₀) aromatic ring or a fused polycyclic aromatic ring with two or more aromatic rings having been fused, provided that Ar₁ is not anthracenyl;
Ar₂ through Ar₄ independently represent a (C₅-C₂₀) aromatic ring or a fused polycyclic aromatic ring with two or more aromatic rings having been fused; and
the aromatic ring or the fused polycyclic aromatic ring with two or more aromatic rings having been fused of Ar₁ through Ar₄ may be further substituted by one or more substituent (s) selected from (C₁-C₂₀)alkyl, (C₁-C₂₀)alkoxy, halogen, tri (C₁-C₂₀)alkylsilyl, tri (C₆-C₂₀)arylsilyl, a (C₅-C₂₀) aromatic ring and a fused polycyclic aromatic ring with two or more aromatic rings having been fused.

5. An organic light emitting device according to claim 4, wherein the organic layer comprises electroluminescent (EL) region, which comprises one or more compound(s) represented by Chemical Formula (1) and one or more EL dopant(s).

6. An organic light emitting device according to claim 5, wherein the EL dopant is selected from the compounds represented by one of Chemical Formulas' (2) to (4): 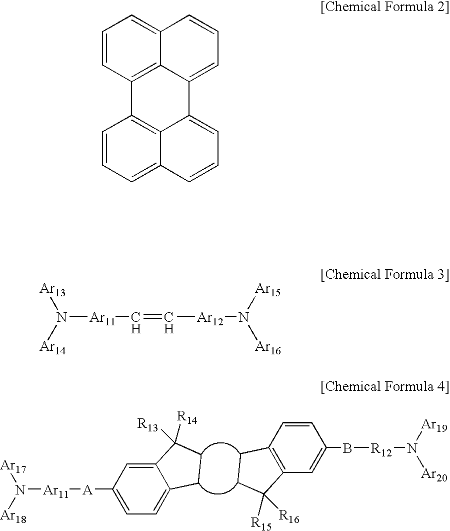
wherein, Ar₁₁ or Ar₁₂ is preferably selected from indenofluorenylene, fluorenylene and spiro-fluorenylene, represented by following chemical formulas:
wherein Ar₁₃ through Ar₁₆ are independently selected from (C₅-C₂₀) aromatic or polycyclic aromatic rings; provided that Ar₁₁ and Ar₁₂ are identical, Ar₁₃ and Ar₁₅ are identical, and Ar₁₄ and Ar₁₆ are identical.
Ar₁₇ through Ar₂₀ independently represent a (C₅-C₂₀) aromatic ring or a fused polycyclic aromatic ring with two or more aromatic rings having been fused;
group 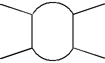
represents 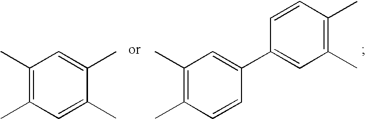
A and B independently represent a chemical bond, or group 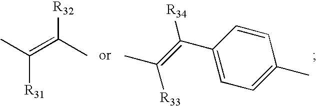
R₁₁ and R₁₂ independently represent a (C₅-C₂₀) aromatic ring or a polycyclic aromatic ring with two or more aromatic rings having been fused;
R₁₃ through R₁₆ independently represent a linear or branched (C₁-C₂₀) alkyl group with or without halogen substituent(s);
R₂₁ through R₂₆ are independently selected from (C₁-C₂₀) alkyl, and phenyl or naphthyl with or without (C₁-C₅)alkyl substituent(s); and
R₃₁ through R₃₄ independently represent hydrogen or a (C₅-C₂₀) aromatic group.

7. An organic light emitting device according to claim 6, wherein the EL dopant is selected from the compounds represented by one of the following formulas: 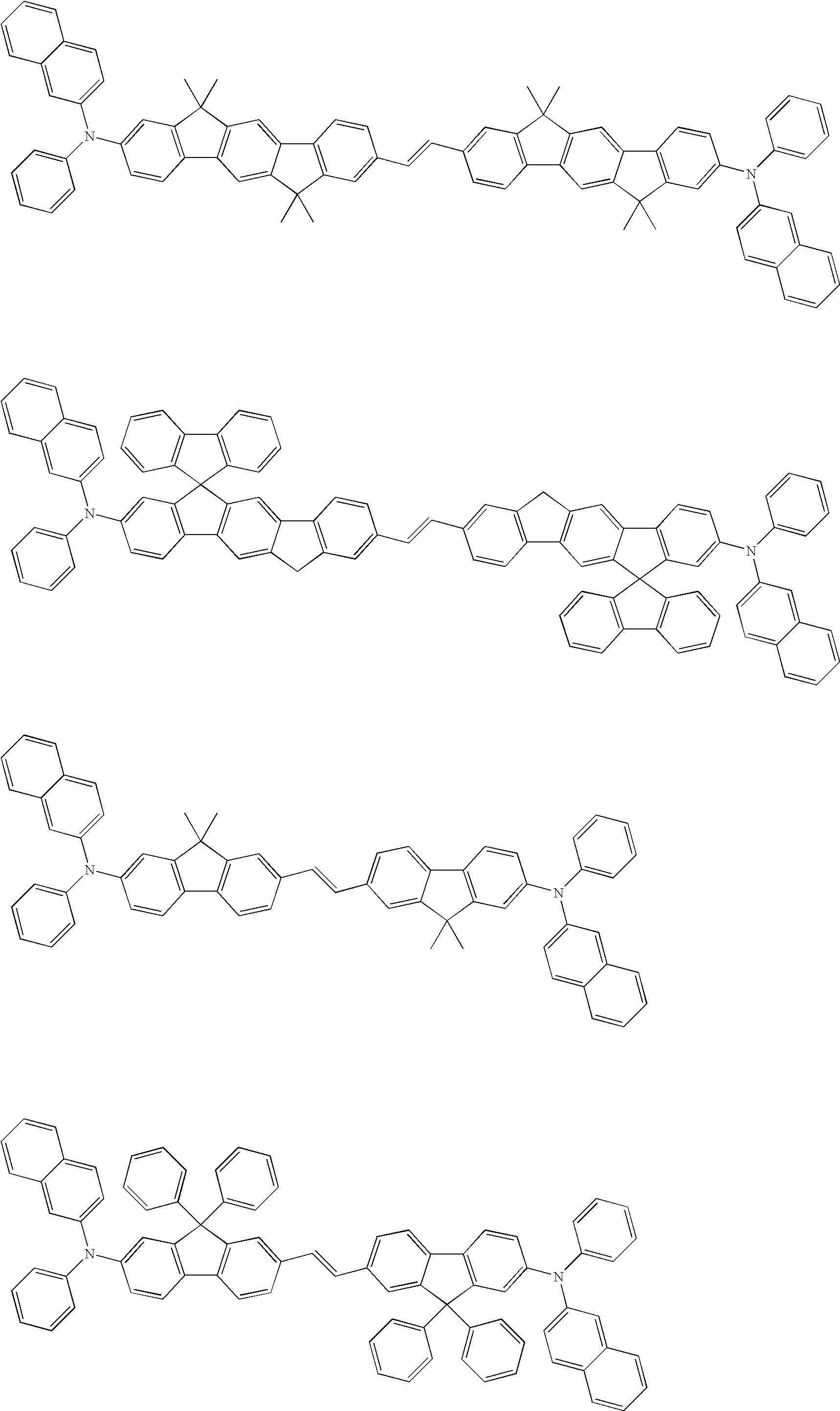 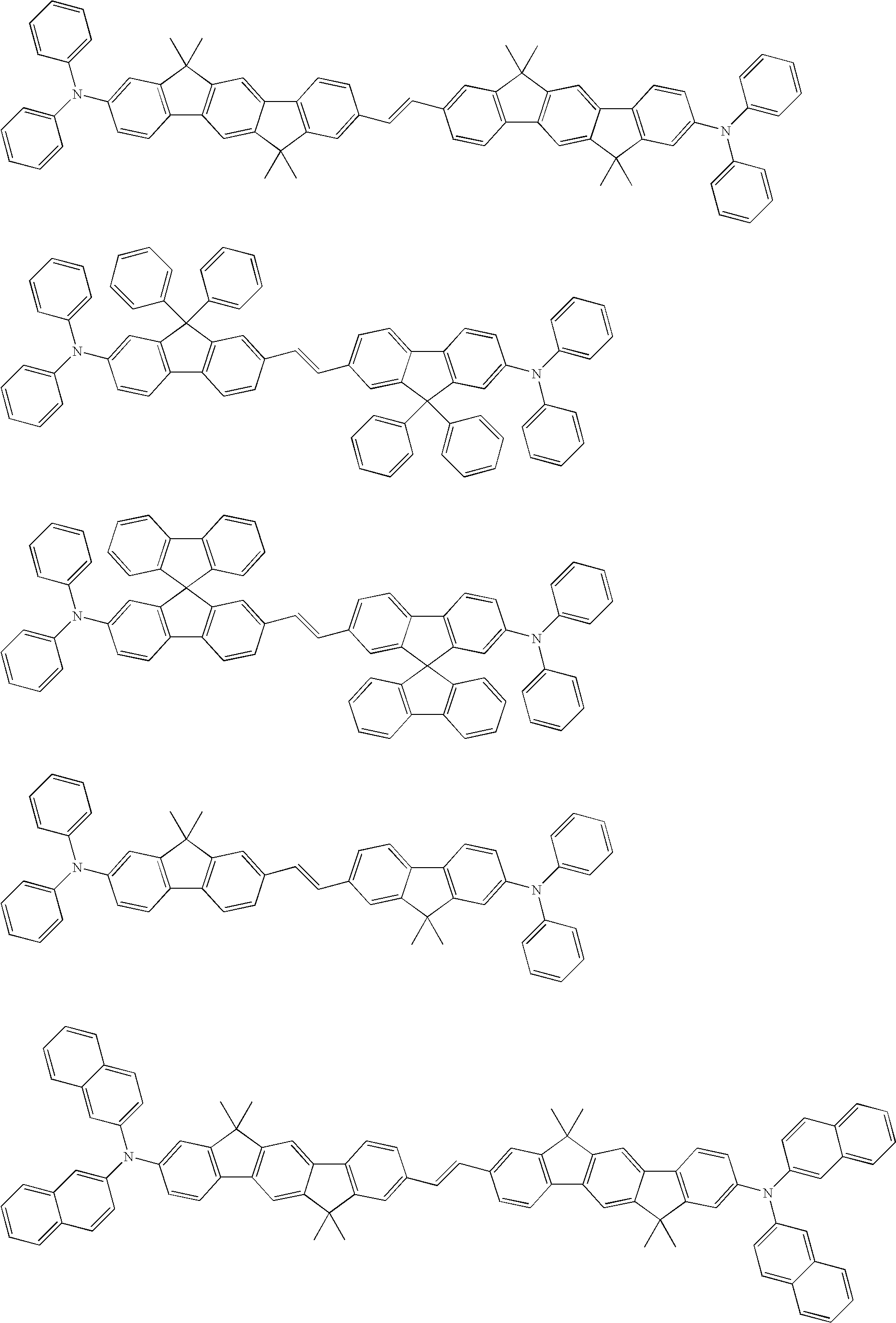 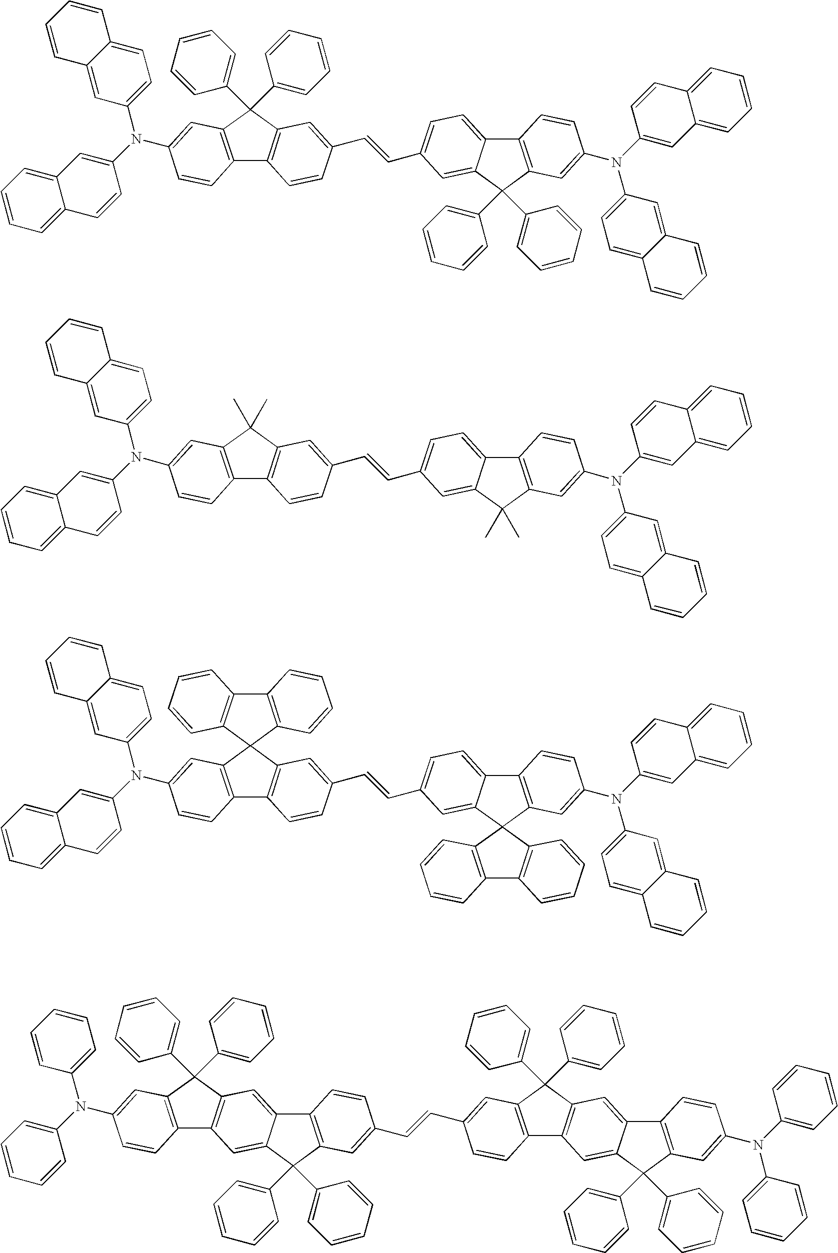 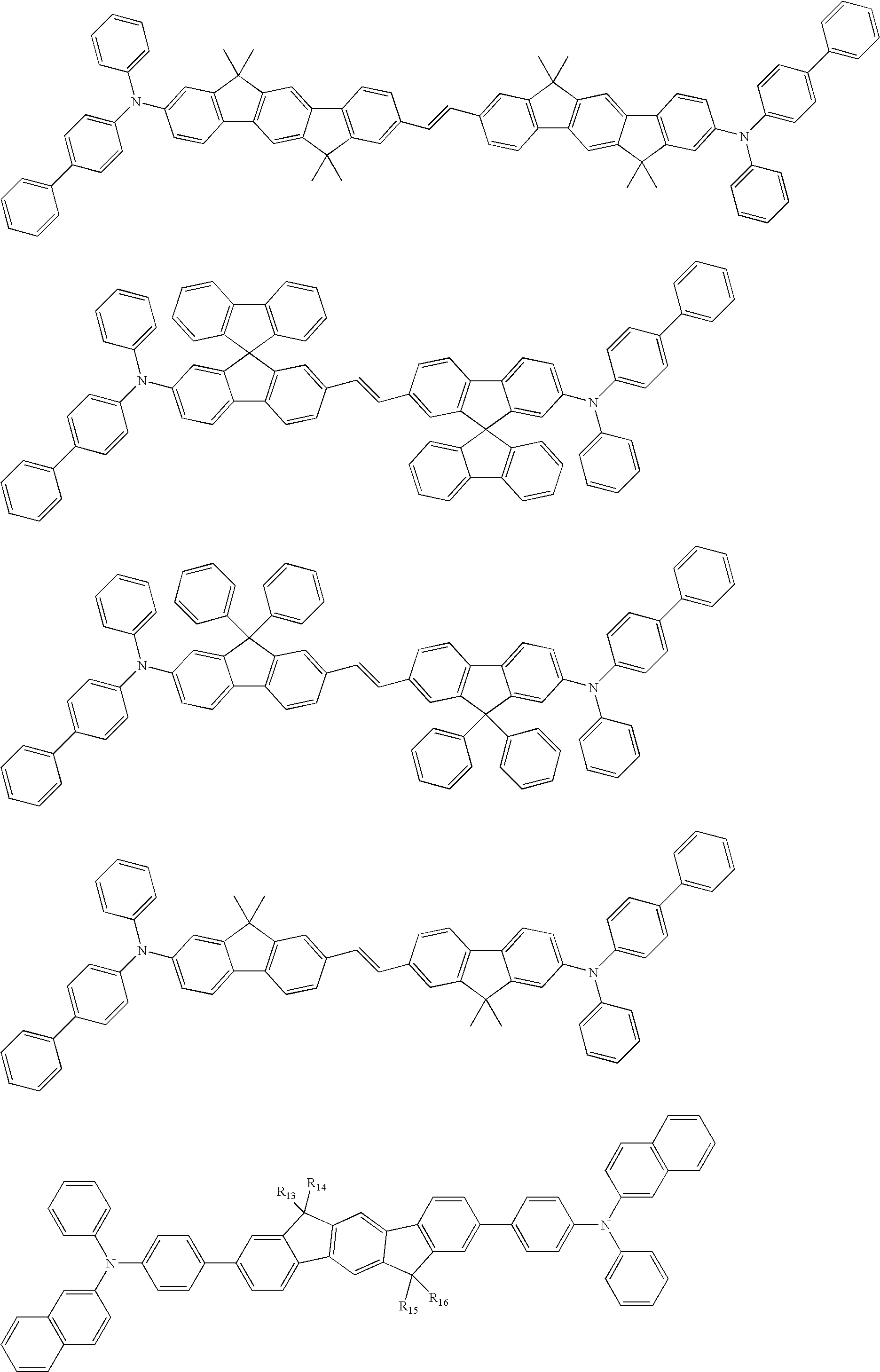 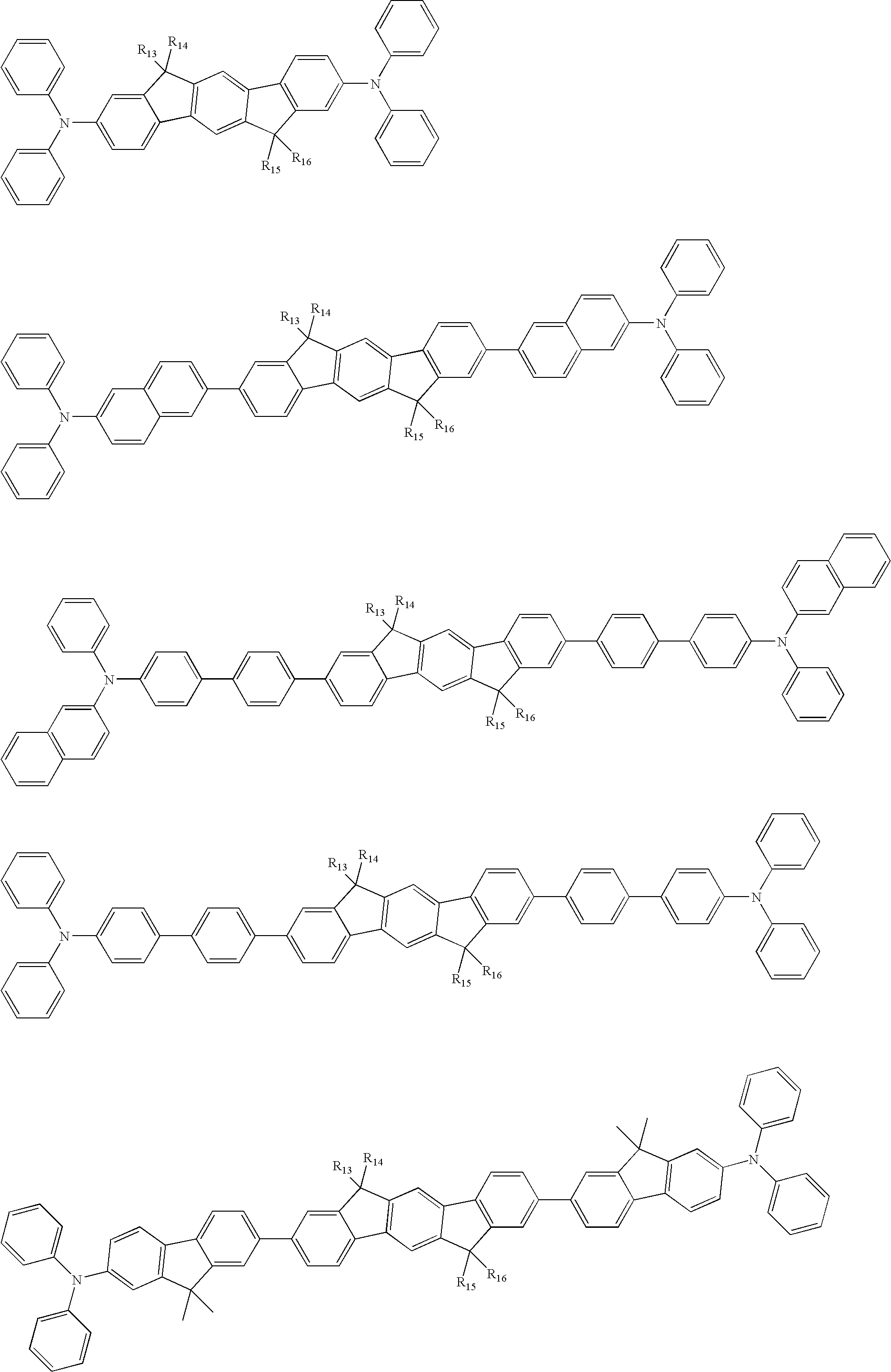 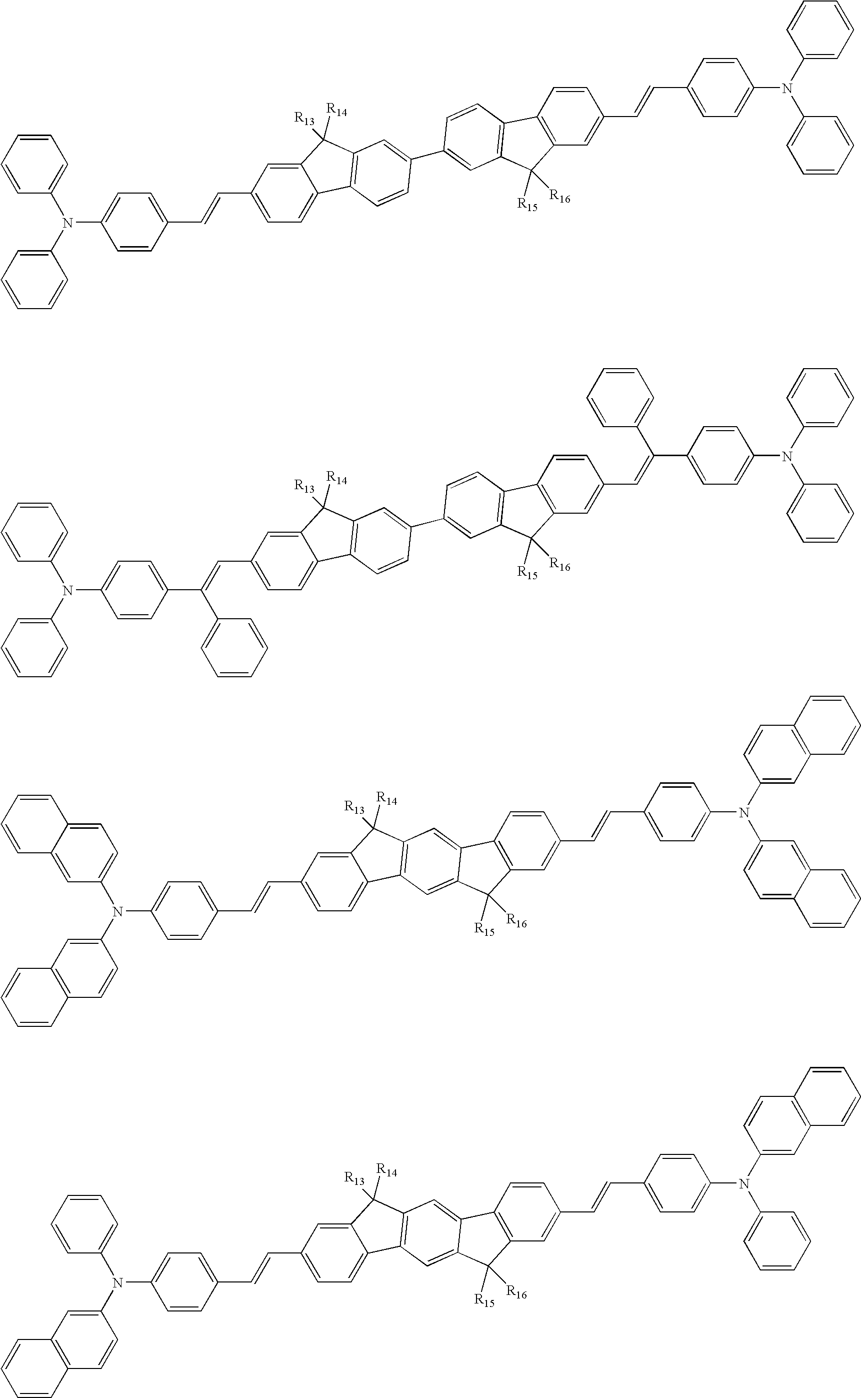 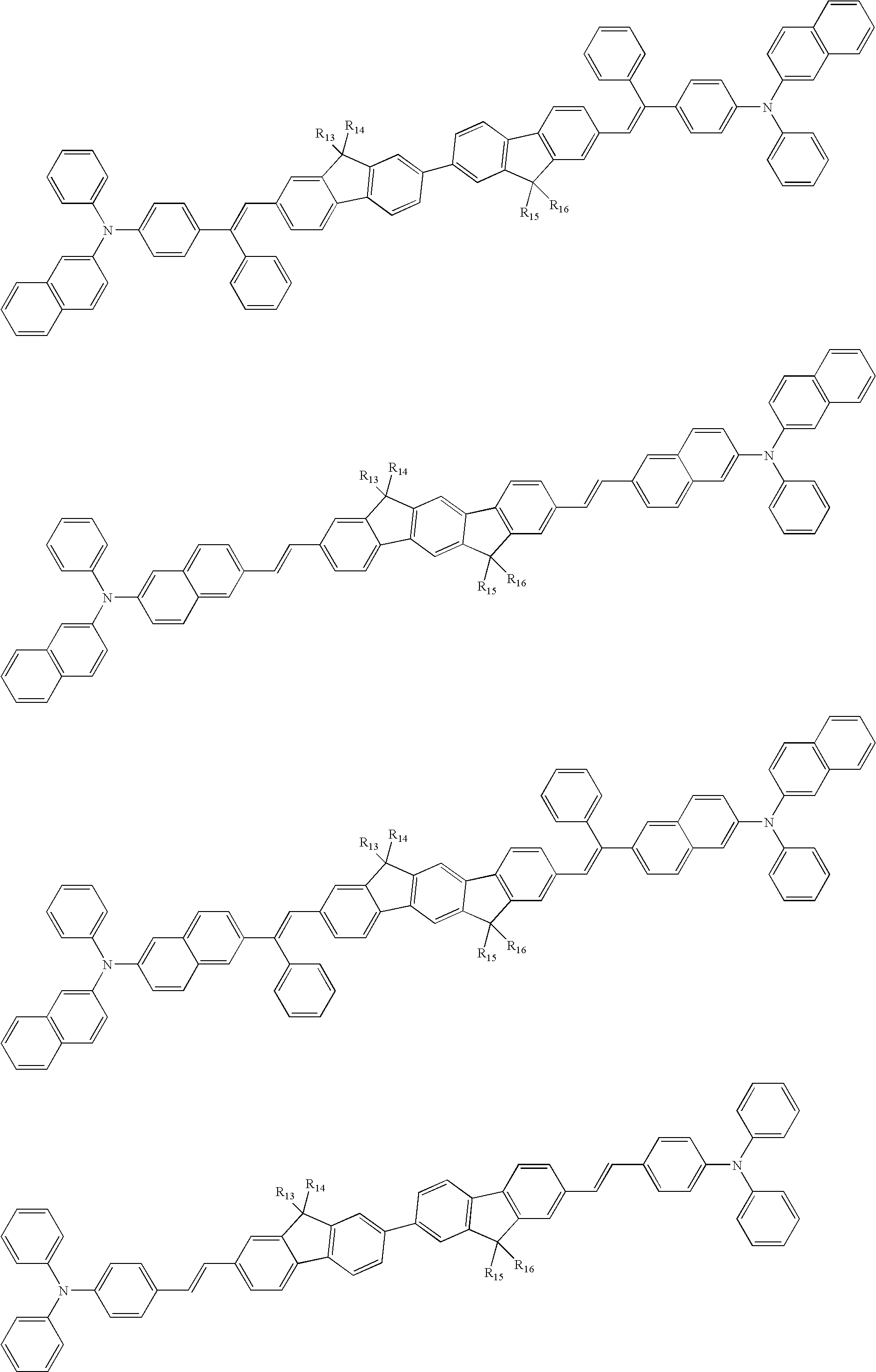 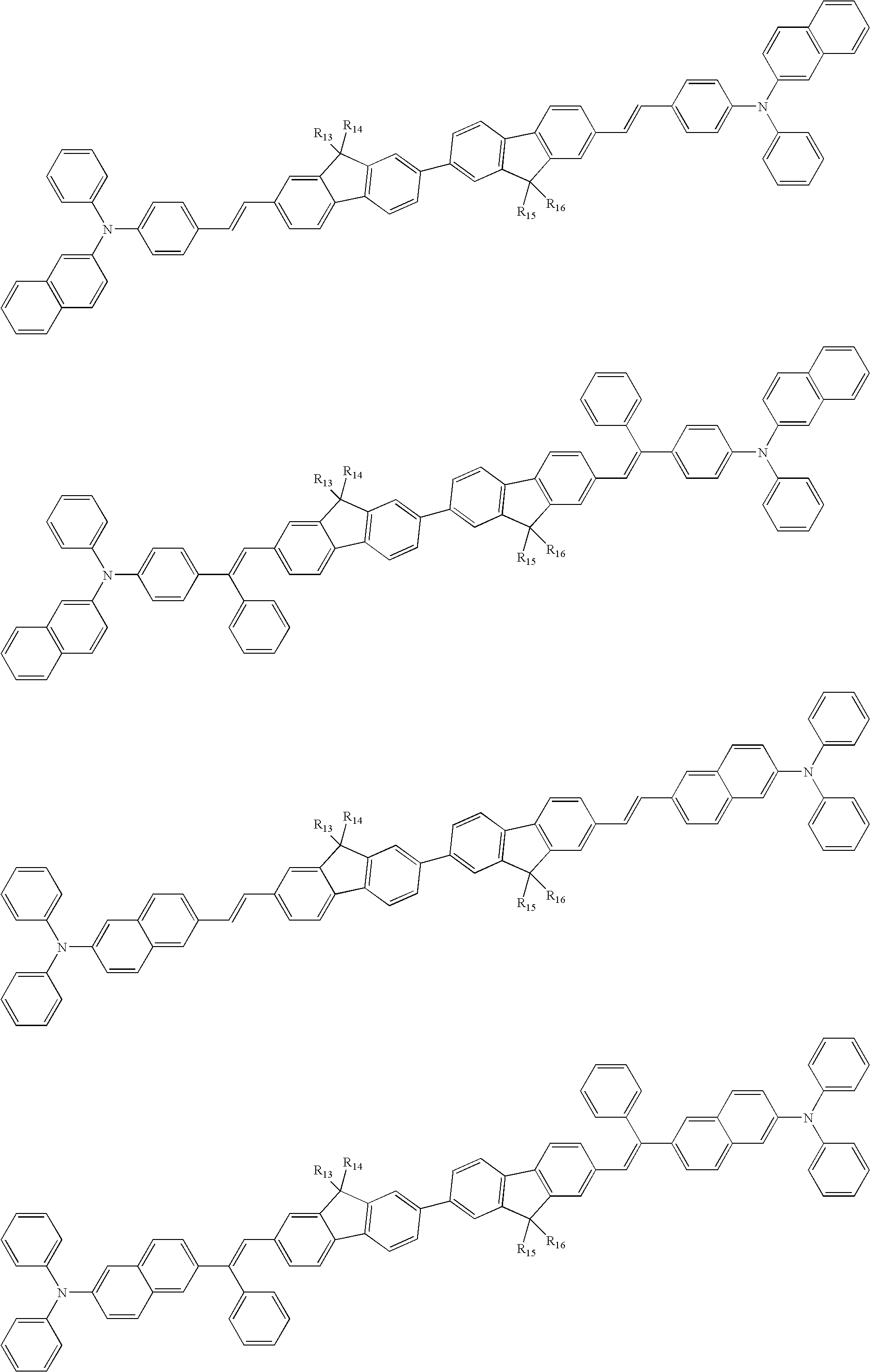 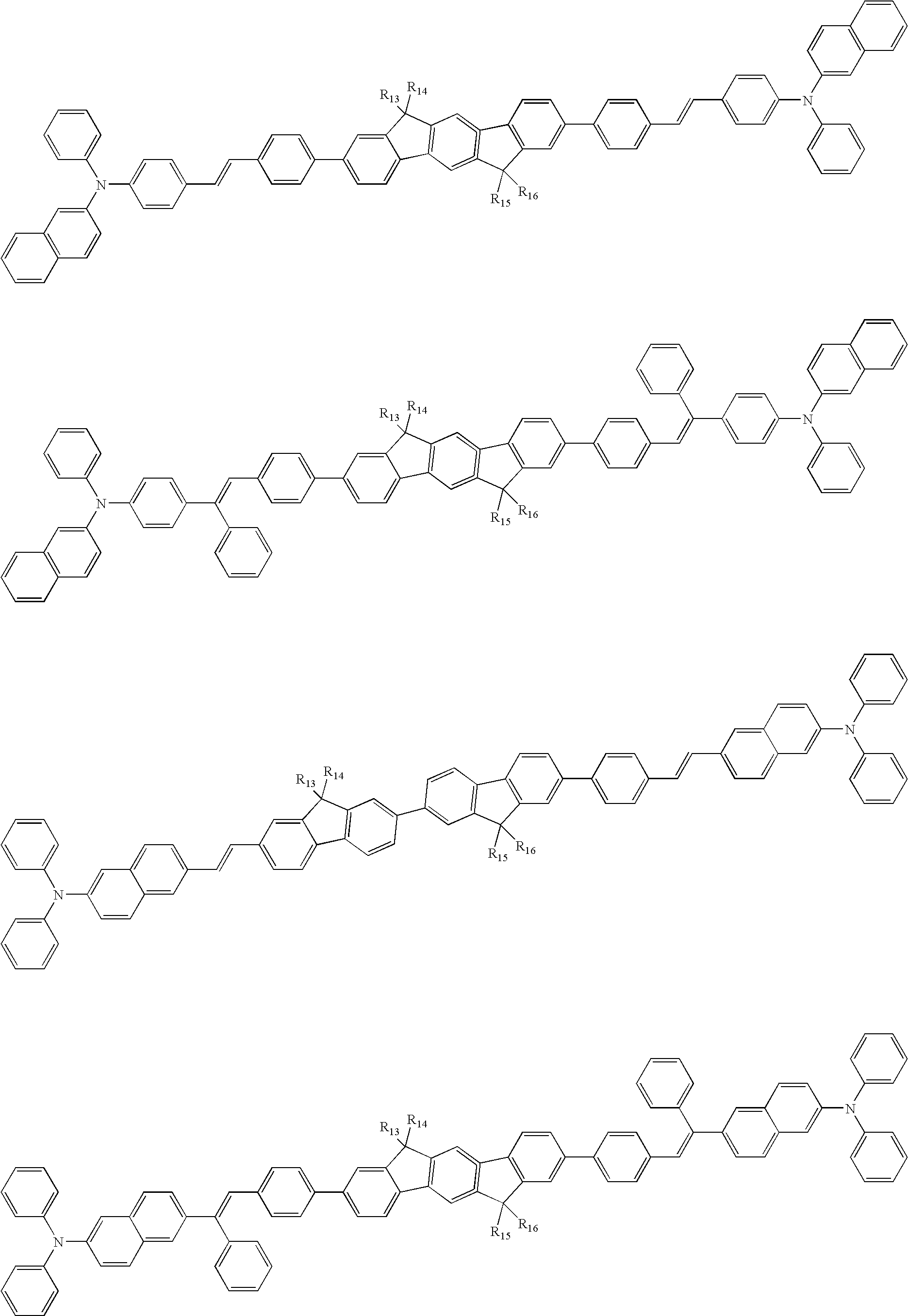 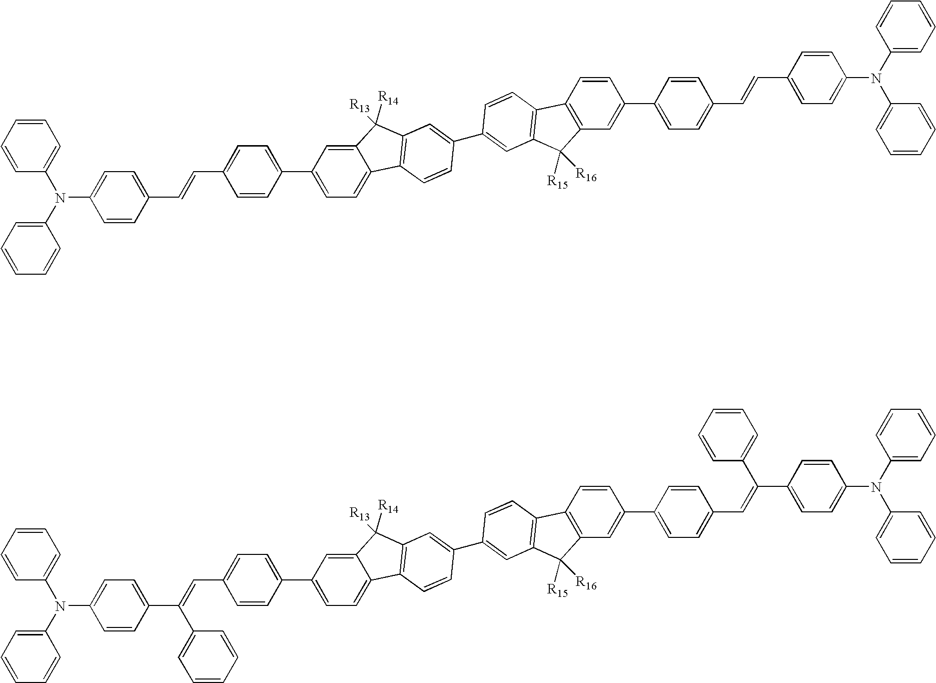
wherein, R₁₃ through R₁₆ represent methyl or ethyl group.

8. An organic light emitting device according to claim 5, wherein the EL dopant is selected from the compounds represented by one of Chemical Formulas (5) to (7): 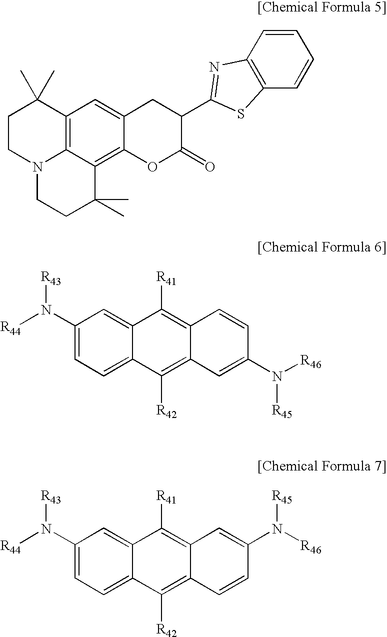
wherein, R₄₁ and R₄₂ independently represent a polycyclic aromatic ring with two more aromatic rings having been fused; R₄₃ through R₄₆ independently represent a (C₅-C₂₀) aromatic ring; and each aromatic ring of R₄₁ through R₄₆ may be further substituted by (C₁-C₂₀) alkyl or (C₅-C₂₀) aryl group(s).

9. An organic light emitting device according to claim 8, wherein the EL dopant is selected from the compounds represented by one of the following formulas: 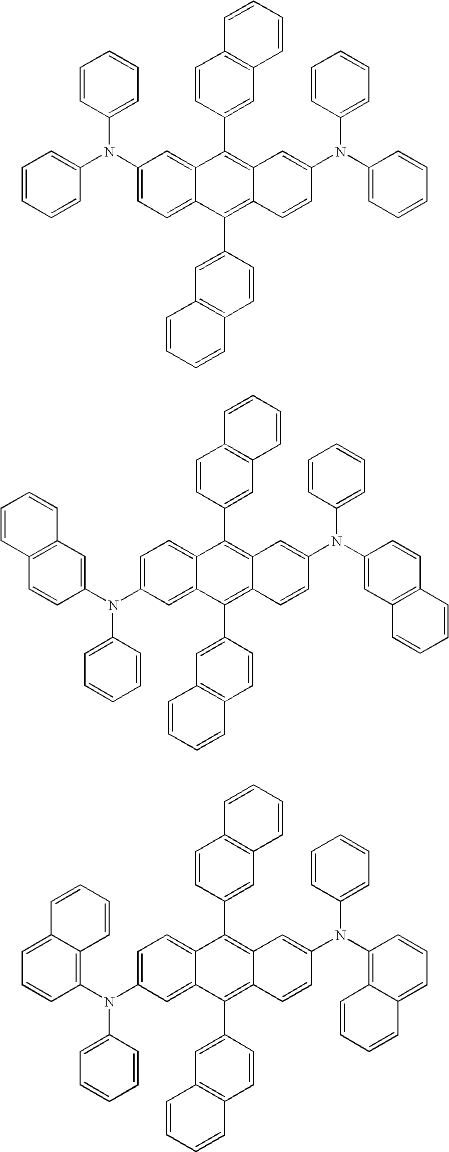 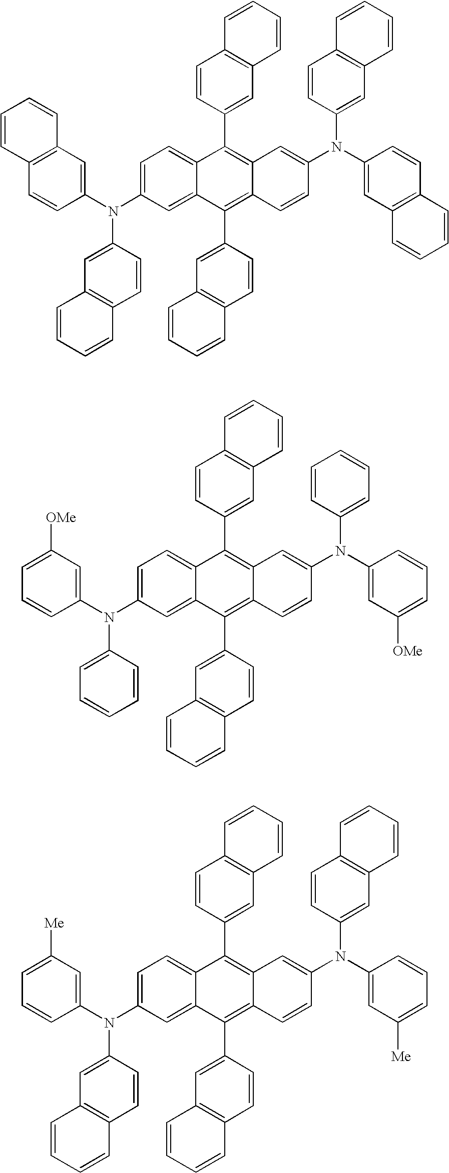 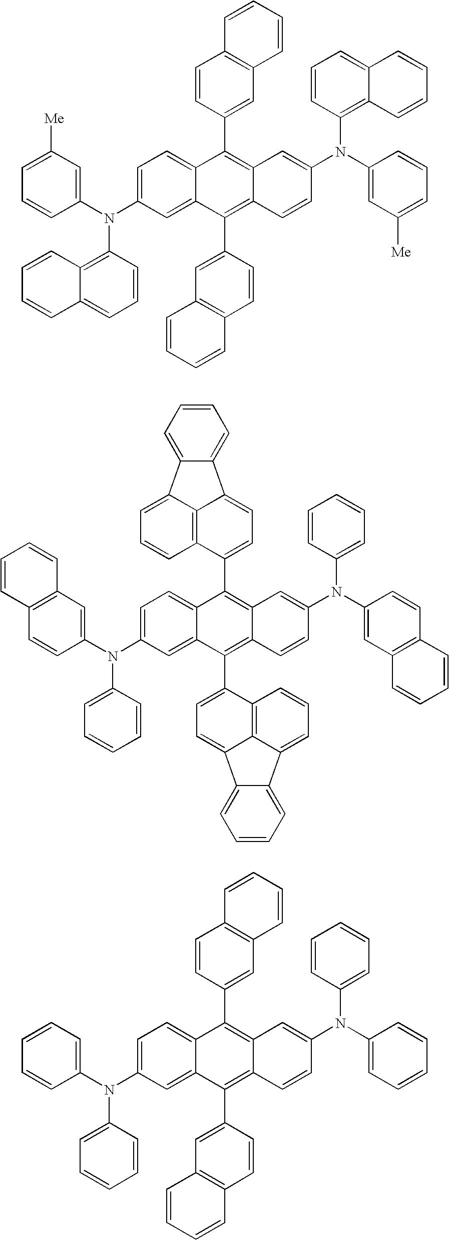 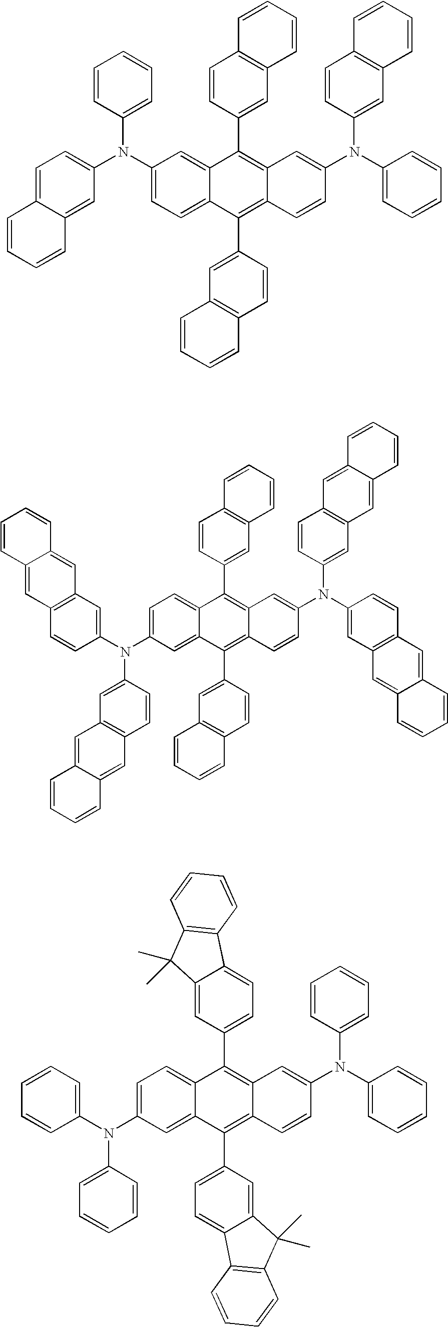 [chemical structure]
